(19) United States
(12) Patent Application Publication
(10) Pub. No.: US 2021/0220331 A1
(43) Pub. Date: Jul. 22, 2021

(54) INHIBITORS OF IRES-MEDIATED PROTEIN SYNTHESIS

(71) Applicants: The Regents of the University of California, Oakland, CA (US); The United State Government represented by the Department of Veterans Affairs, Washington, DC (US)

(72) Inventors: Joseph F. Gera, Lancaster, CA (US); Alan Lichtenstein, Encino, CA (US); Michael E. Jung, Los Angeles, CA (US); Jihye Lee, Yeonsu-gu, Incheon (KR); Brent Holmes, Inglewood, CA (US); Angelica Benavides-Serrato, Grenada Hills, CA (US)

(21) Appl. No.: 16/098,784

(22) PCT Filed: May 3, 2017
(86) PCT No.: PCT/US17/30755

§ 371 Date: Nov. 2, 2018

Related U.S. Application Data
(60) Provisional application No. 62/331,228, filed on May 3, 2016.

(51) Int. Cl. A61K 31/4015; A61K 31/436; A61K 31/519; A61P 35/00; C07D 207/456; C07D 207/452; C07D 209/48; C07D 207/448; C07D 239/54; C07D 217/24; C07D 239/96; C07D 237/32; A61K 31/502; A61K 31/4035; A61K 31/513

(52) U.S. Cl. CPC A61K 31/4015; A61K 31/513; A61K 31/519; A61P 35/00; C07D 207/456; C07D 207/452; C07D 209/48; C07D 207/448; C07D 239/54; C07D 217/24; C07D 239/96; C07D 237/32; A61K 31/502; A61K 31/4035; A61K 31/436

(57) ABSTRACT

This disclosure relates to inhibitors of IRES-mediated protein synthesis, compositions comprising therapeutically effective amounts of these compounds, and methods of using those compounds and compositions in treating hyperproliferative disorders, e.g., cancers. This disclosure also relates to compositions comprising inhibitors of IRES-mediated protein synthesis and mTOR inhibitors, and to methods of treating cancer by conjoint administration of inhibitors of IRES-mediated protein synthesis and mTOR inhibitors.

### STATEMENT REGARDING FEDERALLY SPONSORED RESEARCH OR DEVELOPMENT

This invention was made with government support under Contract No. CA.168700 awarded by The National Institutes of Health. The government has certain rights in the invention.

This work was supported by the U.S. Department of Veterans Affairs, and the Federal Government has certain rights in the invention.

### BACKGROUND OF THE INVENTION

Glioblastoma (GBM) is one of the most common primary malignant brain tumors and median survival is only approximately twelve months (1). The lethality of this tumor is, in part, due the difficulties associated with complete surgical resections and the development of drug resistance (2). As a consequence of EGFR amplification or activating mutation, and PTEN loss (3,4), hyperactivation of the PI3K pathway is frequently seen in nearly 90% of all GBMs (5,6). As a result, a downstream effector, the mechanistic target of rapamycin (mTOR) kinases are often persistently hyperactivated (7). mTOR is a central regulator of metabolism, autophagy and tnRNA translation in the cell and thus, controls tumor cell growth, survival and drug resistance (8,9).

First generation allosteric mTOR inhibitors have failed as monotherapies in the clinic for GBM due to loss of feedback regulation leading to AKT activation (10). Additionally, mTORC2 has been shown to play a critical role in GBM growth, invasion and rapamycin resistance (11,12). These studies have emphasized the potential role of mTOR kinase inhibitors as a potential therapeutic option in the treatment of GBM.

The interrelationships between mTOR signaling complexes suggests the possibility that multiple mechanisms of mTOR. inhibitor resistance exist (13-15). Both allosteric and direct kinase inhibitors of mTOR can activate a transcript-specific protein synthesis salvage pathway maintaining the translation of crucial mRNAs involved in cell-cycle progression resulting in resistance to mTOR therapies (16-18). The activation of this intrinsic pathway is dependent on SAPK2/p38-mediated activation of IRES-dependent initiation of cyclin D1 and c-MYC mRNAs in GBM (19). It has been noted that targeting IRES-dependent c-MYC translation has therapeutic potential; a small molecule inhibitor which blocked c-MYC IRES-mediated translation initiation has been identified (20). However, more effective inhibitors are desired, as well as inhibitors that can act on other pathways involved in IRES-mediated protein synthesis.

### SUMMARY OF THE INVENTION

In one aspect, the present invention provides compounds having the structure of Formula I:

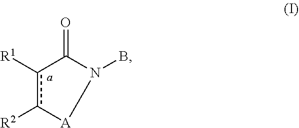

and pharmaceutically acceptable salts and/or prodrugs thereof, wherein the variables are as defined herein. The compounds are typically inhibitors of IRES-mediated protein synthesis. Compounds of formula (I) can be used to treat conditions, such as cancer, as described herein, alone or in combination with an mTOR inhibitor.

The present disclosure also provides compositions (such as pharmaceutical compositions) that comprise the compounds of this disclosure and, optionally, at mTOR inhibitor. The disclosure also includes the use of the compounds or compositions disclosed herein in the manufacture of a medicament for the treatment of one or more of the conditions described herein.

Another aspect of the disclosure provides methods for treating the conditions described herein using the compounds or compositions disclosed herein, including methods for treating cancer in a subject in need thereof.

### BRIEF DESCRIPTION OF THE DRAWINGS

FIGS. 1A-1F show that C11 inhibits both cyclin D1 and c-MYC IRES activity in glioblastoma cells. FIG. 1A: Schematic diagram of generalized dicistronic construct used. FIG. 1B: Schematic diagrams of dicistronic constructs used. FIG. 1C: Relative Renilla and firefly luciferase activities obtained from LN229 GBM cells transfected with the indicated constructs in the absence or presence of the inhibitor C11. FIG. 1D: RNA-pull down assays utilizing biotinylated cyclin D1 or c-MYC IRES RNAs. FIG. 1E: Polysome distributions of cyclin D1, c-MYC and actin mRNAs in LN229 cells in the absence or presence of C11 (50 nM). FIG. 1F: Top panel, LN229 cells were treated with C11 as indicated and RT-PCR splicing analysis for Max exon 5 performed. Middle panel, LN229 cells treated with C11 (50 nM) as indicated, were lysed and immunoprecipitated using either eIF-4E or control IgG antibodies. Bound CCND1 or c-MYC RNAs were detected via RT-PCR. Bottom panel, cyclin D1 and c-MYC protein levels from the indicated GBM cell lines in the absence or presence of C11 at 24 h following treatment.

FIGS. 2A-2E shows the synergistic anti-GBM effects of C11 in combination with mTOR inhibitors. FIG. 2A: Inhibition of mTOR inhibitor-induced IRES activity in LN229 cells. FIG. 2B: Growth inhibition of GBM cell lines following 48 h culture in C11. FIG. 2C: Combination analysis of PP242 and C11 inhibitors in GBM cell lines treated with the indicated doses of PP242 alone or in combination for 48 h, and percent growth relative to control cultures was assessed via XTT assays. FIG. 2D: Cell-cycle phase distributions were determined on the indicated GBM cell lines in the absence or presence of PP242 or C11 as shown. Percent apoptotic cells as determined via Annexin V staining are also shown below each graph. FIG. 2E: Transcripion of cyclin D1 or c-myc with various combinations of PP242 and C11.

FIG. 3 shows a schematic representation of various hnRNP A1 deletion mutations.

FIG. 4 shows binding of either cyclin D1 (top panel) or c-MYC (bottom panel) IRES RNAs to GST-tagged hnRNP A1 mutants in the absence or presence of C11 or IRES-J007 as assayed by filter binding.

FIGS. 5A-5E shows a model for potential binding of IRES inhibitors to UP1. FIG. 5A: The electrostatic surface representation of the crystal structure of UP1 is shown with RNP residues of RRM1 and RRM2 labeled in blue. In the 90°-rotated model, the inhibitor interaction pocket is shown in yellow. The inset is a close-up of C11 and IRES-J007 binding to the potential binding site on UP1. Residues predicted to interact with the inhibitors are labeled. FIG. 5B: Purified GST-tagged wild-type hnRNP A1 (A1) and mutant A1 (4ΔA1) proteins harboring alanine substitutions at all four potential binding sites (120, 123,124 and 171) were added to uncross-linked, C11 and J007-cross-linked beads. Isolated wild-type (A1) and mutant (4ΔA1) proteins were resolved by SDS-PAGE and silver-stained to monitor purity (top panels). The binding of A1 to control, C11 and J007 beads was detected by immunoblotting with GST antibodies (bottom panel). FIG. 5C: Inhibition of IRES activity in cells containing wild-type (A1) and mutant (4ΔA1) proteins. FIG. 5D: Inhibition of basal IRES activity in 293T cells upon treatment with C11 or IRES-J007. FIG. 5E: RNA-pull down assays utilizing biotinylated cyclin D1 or c-MYC IRES RNAs of 293T cell extracts treated with the inhibitors as in FIG. 2C.

FIGS. 6A-6C shows combination effects of PP242 and IRES-J007 on GBM tumor growth in mice. FIG. 6A: Tumor burden of SCID mice implanted with LN229 cells and treated double vehicle, PP242, J007, or combination for ten consecutive days and tumor growth assessed every two days following initiation of treatment (start, day 0). *, P<0.05, significantly different from double vehicle, PP242 (50 mg/kg/d) and J007 (20 mg/kg/d). FIG. 6B: Overall survival of subcutaneous LN229 tumors receiving the indicated treatment schedules. FIG. 6C: Left panel, apoptotic cells were identified by TUNEL assays of sections prepared from harvested tumors at day 12 following initiation of treatment regimens. Middle panel, Cyclin D1 protein levels in tumors. Right panel, c-MYC protein levels in tumors.

FIG. 7 shows Cyclin D1 and c-MYC mRNA translational state in subcutaneous LN229 GBM tumors in response to combination IRES and mTOR inhibitor therapy.

FIG. 8 shows pharmacokinetic parameters for IRES-J007 in mice.

### DETAILED DESCRIPTION

In one aspect, the present disclosure provides a compound of Formula I:

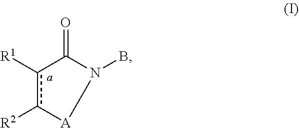

and pharmaceutically acceptable salts and/or prodrugs thereof, wherein:

A is selected from —C(O)—, —C(O)C(R³)₂—, —NR⁴C(O)—, or —C(O)NR⁴—, wherein the right-hand valence is attached to the nitrogen atom of Formula I, preferably —C(O)—;

B is selected from alkyl, aryl, heteroaryl, aralkyl, heteroaralkyl, arylamino, or heteroarylamino, preferably aryl or aralkyl;

R¹ and R² are independently selected from H, alkyl, cycloalkyl, heterocyclyl, aryl, heteroaryl, cyano, halo, hydroxyl, carbonyl, thiocarbonyl, alkoxyl, amino, amido, amidine, imine, sulfhydryl, alkylthio, sulfate, sulfonate, sulfamoyl, sulfonamido, or sulfonyl; or R¹ and R², taken together with the carbon atoms that separate them, complete a cycle or heterocycle having from 4 to 8 atoms in the ring structure;

the bond marked with an ‘a’ is selected from a single or double bond, preferably a double bond;

R³ is selected from H, alkyl, cycloalkyl, heterocyclyl, aryl, heteroaryl, cyano, halo, hydroxyl, carbonyl, thiocarbonyl, alkoxyl, amino, amido, amidine, imine, sulfhydryl, alkylthio, sulfate, sulfonate, sulfamoyl, sulfonamido, or sulfonyl, preferably alkyl.

R⁴ is selected from H, alkyl, cycloalkyl, heterocyclyl, aryl, heteroaryl, carbonyl, thiocarbonyl, or alkoxyl, preferably alkyl.

In certain embodiments, R¹ and R² are not both chloro. In certain embodiments, B is not 2,4-dimethoxybenzyl. In certain embodiments, it is not the case that R¹ and R² are chloro and B is 2,4-dimethoxybenzyl. In certain such embodiments, B is not 4-methoxyphenyl or 4-fluorophenyl. In certain embodiments, B is not substituted or unsubstituted phenyl.

In certain embodiments, R¹ and R² are independently selected from H, alkyl, phenyl, or fluoro. In certain embodiments, R¹ and R² are independently selected from H, C1-6 alkyl, phenyl, or fluoro.

In certain embodiments, R¹ and R², taken together with the carbon atoms that separate them, complete a cyclic or heterocyclic moiety having from 4 to 8 atoms in the ring structure; the cycle or heterocycle is optionally substituted by at least one alkyl, cycloalkyl, heterocyclyl, aryl, heteroaryl, cyano, halo, hydroxyl, carbonyl, thiocarbonyl, alkoxyl, amino, amido, amidine, imine, sulfhydryl, alkylthio, sulfate, sulfonate, sulfamoyl, sulfonamido, or sulfonyl. In certain preferred embodiments, R¹ and R², taken together with the carbon atoms that separate them, complete a substituted or unsubstituted phenyl ring.

In certain embodiments, R³ is selected from H, C1-6 alkyl, or C3-6 cycloalkyl. In certain embodiments, R³ is selected from H, C1-3 alkyl, or C3-4 cycloalkyl. In certain embodiments, R³ is selected from methyl, ethyl, isopropyl, tert-butyl, or cyclopropyl. In certain embodiments, R³ is methyl.

In certain embodiments, R⁴ is selected from H, C1-6 alkyl, or C3-6 cycloalkyl. In certain embodiments, R⁴ is selected from H, C1-3 alkyl, or C3-4 cycloalkyl. In certain embodiments, R⁴ is selected from methyl, ethyl, isopropyl, tert-butyl, or cyclopropyl. In certain embodiments, R⁴ is methyl.

In certain embodiments, the compound of Formula I is represented by one of the following structures:

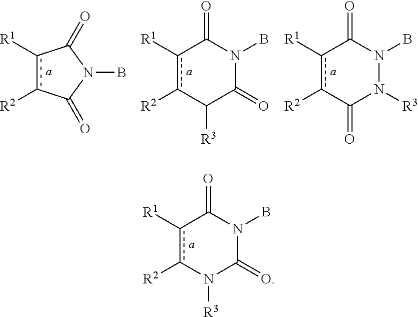

In certain embodiments, the compound of Formula I is represented by one of the following structures:

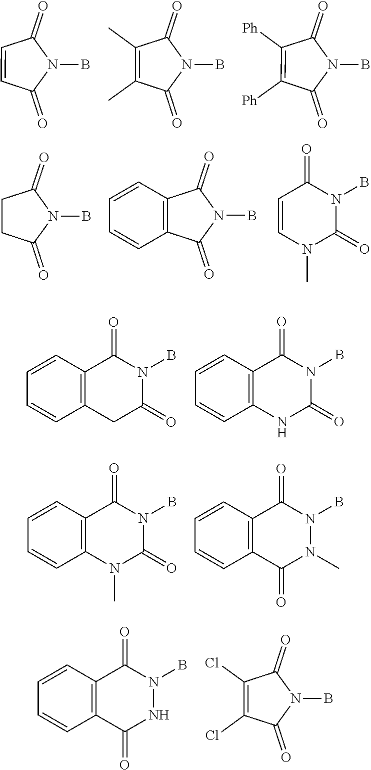

In certain embodiments, B is selected from aryl, heteroaryl, aralkyl, heteroaralkyl, or arylamino, heteroarylamino. In certain embodiments, B is selected from C₆₋₁₀ aryl, C₇₋₁₃ aralkyl, 5-10 member heteroaryl, 6-13 member heteroaralkyl, 6-13 member arylamino, or 6-13 member heteroarylamino; in certain embodiments, B is selected from phenyl, benzyl, phenylamino, and diphenylamino, and may be substituted or unsubstituted. The substituents on B are preferably selected from alkyl, alkoxy, halo, or amino, such as C₁₋₆ alkyl, C₁₋₆ alkoxy, halo, or amino.

In certain embodiments, B is selected from benzyl, 2,4-dimethoxybenzyl, 4-methoxybenzyl, 4-fluorobenzyl, 4-methoxyphenyl, phenylamino, diphenylamino, 4-dimethylaminophenyl.

In certain embodiments, compounds of Formula (I) are selected from the compounds depicted in Table 1, preferably IRES-J007, IRES-J008, or IRES-J009.

TABLE 1
Exemplary Compounds.

| Series A | | Series B | |
|---|---|---|---|
| Compound | Structure | Compound | Structure |
| C11 | 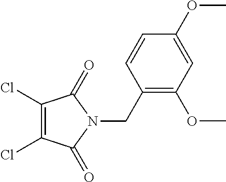 [SMILES: COc1ccc(CN2C(=O)C(Cl)=C(Cl)C2=O)c(OC)c1] | IRES-J000 | 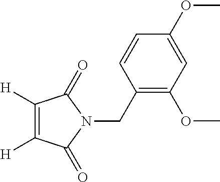 [SMILES: COc1ccc(CN2C(=O)C=CC2=O)c(OC)c1] |
| IRES-J000 | 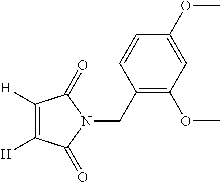 [SMILES: COc1ccc(CN2C(=O)C=CC2=O)c(OC)c1] | IRES-J001 | 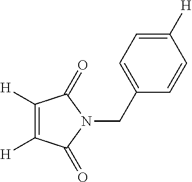 [SMILES: O=C1C=CC(=O)N1Cc1ccccc1] |
| IRES-J004 | 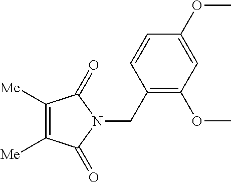 [SMILES: COc1ccc(CN2C(=O)C(C)=C(C)C2=O)c(OC)c1] | IRES-J002 | 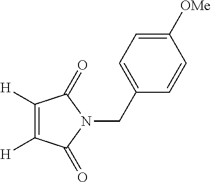 [SMILES: COc1ccc(CN2C(=O)C=CC2=O)cc1] |
| IRES-J005 | 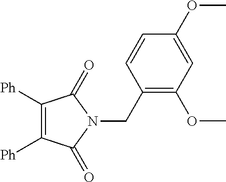 [SMILES: COc1ccc(CN2C(=O)C(c3ccccc3)=C(c3ccccc3)C2=O)c(OC)c1] | IRES-J003 | 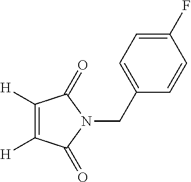 [SMILES: O=C1C=CC(=O)N1Cc1ccc(F)cc1] |
| IRES-J006 | 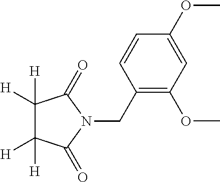 [SMILES: COc1ccc(CN2C(=O)CCC2=O)c(OC)c1] | IRES-J008 | 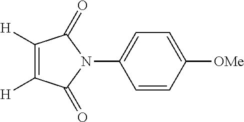 [SMILES: COc1ccc(N2C(=O)C=CC2=O)cc1] |
| IRES-J007 | 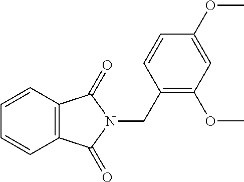 [SMILES: COc1ccc(CN2C(=O)c3ccccc3C2=O)c(OC)c1] | | |
| Series C | | Series D | |
| Compound | Structure | Compound | Structure |
| IRES-J009 | 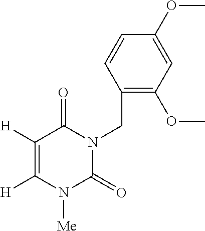 [SMILES: COc1ccc(Cn2c(=O)ccn(C)c2=O)c(OC)c1] | IRES-J010 | 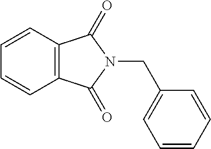 [SMILES: O=C1c2ccccc2C(=O)N1Cc1ccccc1] |
| IRES-J016 | 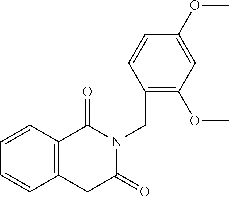 [SMILES: COc1ccc(CN2C(=O)Cc3ccccc3C2=O)c(OC)c1] | IRES-J011 | 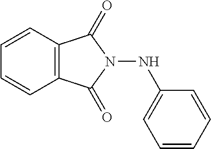 [SMILES: O=C1c2ccccc2C(=O)N1Nc1ccccc1] |
| IRES-J017 | 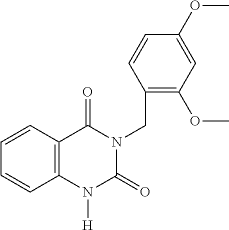 [SMILES: COc1ccc(Cn2c(=O)[nH]c3ccccc3c2=O)c(OC)c1] | IRES-J012 | 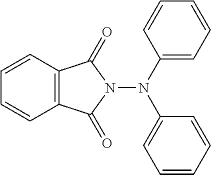 [SMILES: O=C1c2ccccc2C(=O)N1N(c1ccccc1)c1ccccc1] |
| IRES-J018 | 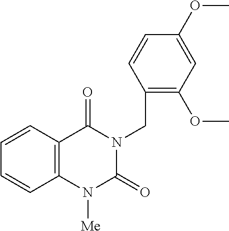 [SMILES: COc1ccc(Cn2c(=O)c3ccccc3n(C)c2=O)c(OC)c1] | IRES-J013 | 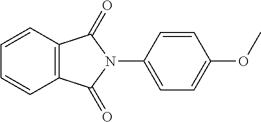 [SMILES: COc1ccc(N2C(=O)c3ccccc3C2=O)cc1] |
| IRES-J019 | 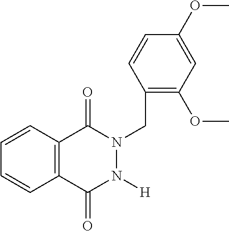 [SMILES: COc1ccc(Cn2[nH]c(=O)c3ccccc3c2=O)c(OC)c1] | IRES-J014 | 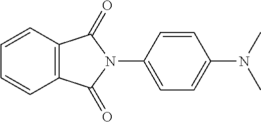 [SMILES: CN(C)c1ccc(N2C(=O)c3ccccc3C2=O)cc1] |
| IRES-J020 | 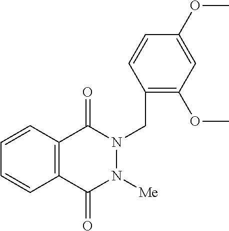 [SMILES: COc1ccc(Cn2c(=O)c3ccccc3c(=O)n2C)c(OC)c1] | IRES-J015 | 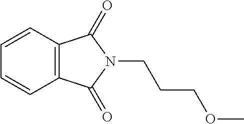 [SMILES: COCCCN1C(=O)c2ccccc2C1=O] |

In certain embodiments, compounds of the invention are prodrugs of the compounds described herein. For example, wherein a hydroxyl in the parent compound is presented as an ester or a carbonate, or a carboxylic acid present in the parent compound is presented as an ester. In certain such embodiments, the prodrug is metabolized to the active parent compound in vivo (e.g., the ester is hydrolyzed to the corresponding hydroxyl or carboxylic acid).

In certain embodiments, compounds of the invention may be racemic. In certain embodiments, compounds of the invention may be enriched in one enantiomer. For example, a compound of the invention may have greater than 30% ee, 40% ee, 50% ee, 60% ee, 70% ee, 80% ee, 90% ee, or even 95% or greater ee. In certain embodiments, compounds of the invention may have more than one stereocenter. In certain such embodiments, compounds of the invention may be enriched in one or more diastereomers. For example, a compound of the invention may have greater than 30% de, 40% de, 50% de, 60% de, 70% de, 80% de, 90% de, or even 95% or greater de.

In certain embodiments, the present invention provides pharmaceutical compositions comprising a compound of Formula I. In certain embodiments, the pharmaceutical compositions further comprise a pharmaceutically acceptable excipient. In certain embodiments, the present invention provides pharmaceutical compositions comprising a compound of Formula I and an mTOR inhibitor, preferably rapamycin or PP242. In certain embodiments, the pharmaceutical compositions may be for use in treating or preventing a condition or disease as described herein.

In certain embodiments, the present invention relates to methods of treatment with a compound of Formula I. In certain embodiments, the therapeutic preparation may be enriched to provide predominantly one enantiomer or isomer of a compound. An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, the therapeutic preparation may be enriched to provide predominantly one diastereomer of a compound. A diastereomerically enriched mixture may comprise, for example, at least 60 mol percent of one diastereomer, or more preferably at least 75, 90, 95, or even 99 mol percent.

In certain embodiments, the present invention provides a pharmaceutical preparation suitable for use in a human patient, comprising any of the compounds shown above, and one or more pharmaceutically acceptable excipients.

Compounds of any of the above structures may be used in the manufacture of medicaments for the treatment of any diseases or conditions disclosed herein.

### Uses of the Compounds and Compositions of the Invention

In certain embodiments, the compounds or compositions of the present invention inhibit IRES-mediated protein synthesis in a cell, such as c-Myc IRES translation and cyclin D1 IRES-dependent initiation. Administration of the compounds of the present invention to a subject can cause the inhibition of those pathways in that subject, including in a neoplasm, cancer, or glioblastoma of the subject.

In certain embodiments, compositions of the present invention comprise mTOR inhibitors. mTOR exists within two complexes, mTORC1 and mTORC2. mTORC1 is sensitive to rapamycin and rapamycin analogs (such as temsirolimus or everolimus) and mTORC2 is largely rapamycin-insensitive. Several mTOR inhibitors have been or are being evaluated in clinical trials for the treatment of cancer. As used herein, the term “mTOR inhibitor” refers to a compound or a ligand that inhibits at least one activity of an mTOR, such as the serine/threonine protein kinase activity on at least one of its substrates (e.g., p70S6 kinase 1, 4E-BP1, AKT/PKB and eEF2). A person skilled in the art can readily determine whether a compound, such as rapamycin or an analogue or derivative thereof, is an mTOR inhibitor. Methods of identifying such compounds or ligands are known in the art. Examples of mTOR inhibitors include, without limitation, rapamycin (sirolimus), rapamycin derivatives, CI-779, everolimus (Certican™), ABT-578, tacrolimus (FK 506), ABT-578, AP-23675, BEZ-235, OSI-027, QLT-0447, ABI-009, BC-210, salirasib, TAFA-93, deforolimus (AP-23573), temsirolimus (Torisel™), 2-(4-Amino-1-isopropyl-1H-pyrazolo[3,4-d]pyrimidin-3-yl)-1H-indol-5-ol (PP242) and AP-23841.

In certain embodiments, compounds or compositions of the present invention are used to treat cancer. In some embodiments, the cancer is a solid tumor. In some embodiments, the cancer is not a solid tumor. In certain embodiments, the cancer is ovarian cancer; endometrial cancer, such as endometrial carcinoma; breast cancer; colon cancer; brain cancer, such as glioblastoma; neuroblastoma; lung cancer, such as lung carcinoma or small-cell lung carcinoma; skin cancer, such as melanoma; renal cancer, such as renal cell carcinoma; liver cancer, such as hepatocellular carcinoma; prostate cancer; head or neck carcinoma; pancreatic cancer, such as pancreatic carcinoma; thyroid cancer, such as thyroid carcinoma; leukemia; lymphoma; multiple myeloma; rhabdomyosarcoma; osteosarcoma, or Ewing sarcoma. Preferably, the cancer is glioblastoma. In some embodiments, the subject has Peutz Jeghers cancer prone syndrome or tuberous sclerosis syndrome. In certain embodiments, the compounds or compositions of the present invention are used conjointly with an mTOR inhibitor, such as those described herein, preferably rapamycin or PP242.

In certain embodiments, the invention provides methods of treating cancer comprising administering a compound or composition as disclosed herein to a subject. In some embodiments, the subject is a mammal. For example, the subject may be a mouse or a human. In certain embodiments, the cancer is ovarian cancer; endometrial cancer, such as endometrial carcinoma; breast cancer; colon cancer; brain cancer, such as glioblastoma; neuroblastoma; lung cancer, such as lung carcinoma or small-cell lung carcinoma; skin cancer, such as melanoma; renal cancer, such as renal cell carcinoma; liver cancer, such as hepatocellular carcinoma; prostate cancer; head or neck carcinoma; pancreatic cancer, such as pancreatic carcinoma; thyroid cancer, such as thyroid carcinoma; leukemia; lymphoma; multiple myeloma; rhabdomyosarcoma; osteosarcoma, or Ewing sarcoma. Preferably, the cancer is glioblastoma. In some embodiments, the subject has Peutz Jeghers cancer prone syndrome or tuberous sclerosis syndrome.

In certain embodiments, the compounds or compositions of the present invention are administered conjointly with an mTOR inhibitor, such as those described herein, preferably rapamycin or PP242.

In certain embodiments, compounds or compositions of the present invention are used to inhibit IRES-mediated protein synthesis within a cell. In some embodiments, the compounds or compositions inhibit c-Myc IRES translation or cyclin D1 IRES-dependent initiation. In some embodiments, the compounds or compositions that are used include an mTOR inhibitor, such as those described herein, preferably rapamycin or PP242.

In certain embodiments, the invention provides methods of inhibiting IRES-mediated protein synthesis within a cell by contacting the cell with a compound or composition of the present invention. In some embodiments, inhibiting IRES-mediated protein synthesis comprises inhibiting c-Myc IRES translation or cyclin D1 IRES-dependent initiation. In some embodiments, the cell is also contacted with an mTOR inhibitor, such as those described herein, preferably rapamycin or PP242.

### Compositions and Modes of Administration

In some embodiments (such as the uses described above), the compounds of the disclosure are formulated into pharmaceutical compositions for administration to subjects (such as human subjects) in a biologically compatible form suitable for administration in vivo. Accordingly, in another aspect, the present invention provides a pharmaceutical composition comprising a compound of the disclosure in admixture with a suitable diluent or carrier. Such a composition is useful for treating the conditions described herein.

The compositions containing the compounds of the disclosure can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

The compounds of this invention may be used in treating the conditions described herein, in the form of the free base, salts (preferably pharmaceutically acceptable salts), solvates, hydrates, prodrugs, isomers, or mixtures thereof. All forms are within the scope of the disclosure. Acid addition salts may be formed and provide a more convenient form for use; in practice, use of the salt form inherently amounts to use of the base form. The acids which can be used to prepare the acid addition salts include preferably those which produce, when combined with the free base, pharmaceutically acceptable salts, that is, salts whose anions are non-toxic to the subject organism in pharmaceutical doses of the salts, so that the beneficial properties inherent in the free base are not vitiated by side effects ascribable to the anions. Although pharmaceutically acceptable salts of the basic compounds are preferred, all acid addition salts are useful as sources of the free base form even if the particular salt per se is desired only as an intermediate product as, for example, when the salt is formed only for the purposes of purification and identification, or when it is used as an intermediate in preparing a pharmaceutically acceptable salt by ion exchange procedures.

Pharmaceutically acceptable salts within the scope of the disclosure include those derived from the following acids; mineral acids such as hydrochloric acid, sulfuric acid, phosphoric acid and sulfamic acid; and organic acids such as acetic acid, citric acid, lactic acid, tartaric acid, malonic acid, methanesulfonic acid, ethanesulfonic acid, benzenesulfonic acid, p-toluenesulfonic acid, cyclohexylsulfamic acid, quinic acid, and the like.

In accordance with the methods of the disclosure, the described compounds may be administered to a patient in a variety of forms depending on the selected route of administration, as will be understood by those skilled in the art. The compositions of the disclosure may be administered orally or parenterally.

Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

In certain embodiments, pharmaceutical compositions suitable for parenteral administration may comprise the compound of the present disclosure in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

A composition comprising a compound of the present disclosure may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

In certain embodiments of the disclosure, compositions comprising a compound of the present disclosure can be administered orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and the like, each containing a predetermined amount of the compound of the present disclosure as an active ingredient.

In solid dosage forms for oral administration (capsules, tablets, pills, dragees, powders, granules, and the like), one or more compositions comprising the compound of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the compound of the present disclosure, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, salts and/or prodrugs thereof, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

A person skilled in the art would know how to prepare suitable formulations. Conventional procedures and ingredients for the selection and preparation of suitable formulations are described, for example, in Remington's Pharmaceutical Sciences (1990-18th edition) and in The United States Pharmacopeia: The National Formulary (USP 24 NF19) published in 1999.

The pharmaceutical forms suitable for injectable use include sterile aqueous solutions or dispersion and sterile powders for the extemporaneous preparation of sterile injectable solutions or dispersions.

The compounds of the disclosure may be administered to a subject in need thereof alone or in combination with pharmaceutically acceptable carriers, as noted above, the proportion of which is determined by the solubility and chemical nature of the compound, chosen route of administration and standard pharmaceutical practice.

The dosage of the compounds and/or compositions of the disclosure can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. The compounds of the disclosure may be administered initially in a suitable dosage that may be adjusted as required, depending on the clinical response. To calculate the human equivalent dose (HED) from a dosage used in the treatment of age-dependent cognitive impairment in rats, the formula HED (mg/kg)=rat dose (mg/kg)×0.16 may be employed (see Estimating the Safe Starting Dose in Clinical Trials for Therapeutics in Adult Healthy Volunteers, December 2002, Center for Biologics Evaluation and Research). For example, using that formula, a dosage of 10 mg/kg in rats is equivalent to 1.6 mg/kg in humans. This conversion is based on a more general formula HED=animal dose in mg/kg×(animal weight in kg/human weight in kg)^{0.33} Similarly, to calculate the HED from a dosage used in the treatment in mouse, the formula HED (mg/kg)=mouse dose (mg/kg)×0.08 may be employed (see Estimating the Safe Starting Dose in Clinical Trials for Therapeutics in Adult Healthy Volunteers, December 2002, Center for Biologics Evaluation and Research).

The compounds and/or compositions of the disclosure (including compositions with and without mTOR inhibitors) can be used alone or conjointly with other therapeutic agents, or in combination with other types of treatment (which other types of treatment may or may not inhibit IRES-mediated protein synthesis or mTOR) for treating cell proliferative disorders. For example, these other therapeutically useful agents may be administered in a single formulation, simultaneously or sequentially with the compound of the present disclosure according to the methods of the disclosure.

There are various examples of other types of treatment for cell proliferative disorders currently used to treat different types of cancers. In a particular aspect of the present invention, the compounds and/or compositions of the disclosure may be used in combination with other therapies and therapeutics to treat leukemia.

In some embodiments, the method of treating or preventing cancer, such as those described above, may comprise administering a compound or composition of the disclosure conjointly with one or more other chemotherapeutic agent(s). Chemotherapeutic agents that may be conjointly administered with compounds or compositions of the disclosure include: aminoglutethimide, amsacrine, anastrozole, asparaginase, bcg, bicalutamide, bleomycin, bortezomib, buserelin, busulfan, campothecin, capecitabine, carboplatin, carfilzomib, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, octreotide, oxaliplatin, paclitaxel, pamidronate, pentostatin, perifosine, plicamycin, pomalidomide, porfimer, procarbazine, raltitrexed, rituximab, sorafenib, streptozocin, sunitinib, suramin, tamoxifen, temozolomide, temsirolimus, teniposide, testosterone, thalidomide, thioguanine, thiotepa, titanocene dichloride, topotecan, trastuzumab, tretinoin, vinblastine, vincristine, vindesine, and vinorelbine. In certain embodiments of the methods of the disclosure described herein, the chemotherapeutic agent conjointly administered with compounds of the disclosure is a taxane chemotherapeutic agent, such as paclitaxel or docetaxel.

Many combination therapies have been developed for the treatment of cancer. In certain embodiments, compounds or compositions of the disclosure may be conjointly administered with a combination therapy. Examples of combination therapies with which compounds of the disclosure may be conjointly administered are included in Table 2.

TABLE 2
Exemplary combinatorial therapies for the treatment of cancer.

| Name | Therapeutic agents |
|---|---|
| ABV | Doxorubicin, Bleomycin, Vinblastine |
| ABVD | Doxorubicin, Bleomycin, Vinblastine, |
| | Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, |
| | Procarbazine, Prednisone |
| BEACOPP | Bleomycin, Etoposide, Doxorubicin, |
| | Cyclophosphamide, Vincristine, Procarbazine, |
| | Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, |
| | Prednisone, Asparaginase |
| CAMP | Cyclophosphamide, Doxorubicin, Methotrexate, |
| | Procarbazine |
| CAP | Cyclophosphamide, Doxorubicin, Cisplatin |
| CaT | Carboplatin, Paclitaxel |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, |
| | with or without/Bleomycin |
| CEV | Cyclophosphamide, Etoposide, Vincristine |
| CF | Cisplatin, Fluorouracil or Carboplatin |
| | Fluorouracil |
| CHAP | Cyclophosphamide or Cyclophosphamide, |
| | Altretamine, Doxorubicin, Cisplatin |
| ChlVPP | Chlorambucil, Vinblastine, Procarbazine, |
| | Prednisone |
| CHOP | Cyclophosphamide, Doxorubicin, Vincristine, |
| | Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, |
| | Prednisone |
| CMFVP | Cyclophosphamide, Methotrexate, Fluorouracil, |
| | Vincristine, Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, |
| | Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, |
| | Leucovorin, Cytarabine |
| COMP | Cyclophosphamide, Vincristine, Methotrexate, |
| | Prednisone |
| Cooper Regimen | Cyclophosphamide, Methotrexate, Fluorouracil, |
| | Vincristine, Prednisone |
| COP | Cyclophosphamide, Vincristine, Prednisone |
| COPE | Cyclophosphamide, Vincristine, Cisplatin, |
| | Etoposide |
| COPP | Cyclophosphamide, Vincristine, Procarbazine, |
| | Prednisone |
| CP(Chronic | Chlorambucil, Prednisone |
| lymphocytic leukemia) | |
| CP (Ovarian Cancer) | Cyclophosphamide, Cisplatin |
| CT | Cisplatin, Paclitaxel |
| CVD | Cisplatin, Vinblastine, Dacarbazine |
| CVI | Carboplatin, Etoposide, Ifosfamide, Mesna |
| CVP | Cyclophosphamide, Vincristine, Prednisone |
| CVPP | Lomustine, Procarbazine, Prednisone |
| CYVADIC | Cyclophosphamide, Vincristine, Doxorubicin, |
| | Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, |
| | Fluorouracil |
| ICE-T | Ifosfamide, Carboplatin, Etoposide, |
| | Paclitaxel, Mesna |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, |
| | Prednisone, Melphalan |
| MAC-III | Methotrexate, Leucovorin, Dactinomycin, |
| | Cyclophosphamide |
| MACC | Methotrexate, Doxorubicin, Cyclophosphamide, |
| | Lomustine |
| MACOP-B | Methotrexate, Leucovorin, Doxorubicin, |
| | Cyclophosphamide, Vincristine, Bleomycin, |
| | Prednisone |
| MAID | Mesna, Doxorubicin, Ifosfamide, Dacarbazine |
| m-BACOD | Bleomycin, Doxorubicin, Cyclophosphamide, |
| | Vincristine, Dexamethasone, Methotrexate, |
| | Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, |
| | Prednisone |
| MOPP/ABV | Mechlorethamine, Vincristine, Procarbazine, |
| | Prednisone, Doxorubicin, Bleomycin, Vinblastine |
| MP (multiple | Melphalan, Prednisone |
| myeloma) | |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, |
| | Prednisone |
| MTX-CDDPAdr | Methotrexate, Leucovorin, Cisplatin, Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, |
| | Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PC | Paclitaxel, Carboplatin or Paclitaxel, Cisplatin |
| PCV | Lomustine, Procarbazine, Vincristine |
| PE | Paclitaxel, Estramustine |
| PFL | Cisplatin, Fluorouracil, Leucovorin |
| POC | Prednisone, Vincristine, Lomustine |
| ProMACE | Prednisone, Methotrexate, Leucovorin, |
| | Doxorubicin, Cyclophosphamide, Etoposide |
| ProMACE/cytaBOM | Prednisone, Doxorubicin, Cyclophosphamide, |
| | Etoposide, Cytarabine, Bleomycin, Vincristine, |
| | Methotrexate, Leucovorin, Cotrimoxazole |
| PRoMACE/MOPP | Prednisone, Doxorubicin, Cyclophosphamide, |
| | Etoposide, Mechlorethamine, Vincristine, |
| | Procarbazine, Methotrexate, Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, |
| | Asparaginase |
| SMF | Streptozocin, Mitomycin, Fluorouracil |
| TAD | Mechlorethamine, Doxorubicin, Vinblastine, |
| | Vincristine, Bleomycin, Etoposide, Prednisone |
| TCF | Paclitaxel, Cisplatin, Fluorouracil |
| TIP | Paclitaxel, Ifosfamide, Mesna, Cisplatin |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, |
| | Cisplatin, Bleomycin |
| VAC | Vincristine, Dactinomycin, Cyclophosphamide |
| VACAdr | Vincristine, Cyclophosphamide, Doxorubicin, |
| | Dactinomycin, Vincristine |
| VAD | Vincristine, Doxorubicin, Dexamethasone |
| VATH | Vinblastine, Doxorubicin, Thiotepa, |
| | Flouxymesterone |
| VBAP | Vincristine, Carmustine, Doxorubicin, Prednisone |
| VBCMP | Vincristine, Carmustine, Melphalan, |
| | Cyclophosphamide, Prednisone |
| VC | Vinorelbine, Cisplatin |
| VCAP | Vincristine, Cyclophosphamide, Doxorubicin, |
| | Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, |
| | Prednisone |
| VP | Etoposide, Cisplatin |
| V-TAD | Etoposide, Thioguanine, Daunorubicin, |
| | Cytarabine |
| 5 + 2 | Cytarabine, Daunorubicin, Mitoxantrone |
| 7 + 3 | Cytarabine with/, Daunorubicin or Idarubicin or |
| | Mitoxantrone |
| “8 in 1” | Methylprednisolone, Vincristine, Lomustine, |
| | Procarbazine, Hydroxyurea, Cisplatin, |
| | Cytarabine, Dacarbazine |

In certain embodiments, a compound or composition of the disclosure may be conjointly administered with non-chemical methods of cancer treatment. In certain embodiments, a compound or composition of the disclosure may be conjointly administered with radiation therapy. In certain embodiments, a compound or composition of the disclosure may be conjointly administered with surgery, with thermoablation, with focused ultrasound therapy, with cryotherapy, or with any combination of these.

In certain embodiments, different compounds of the disclosure may be conjointly administered with one or more other compounds of the disclosure. Moreover, such combinations may be conjointly administered with other therapeutic agents, such as other agents suitable for the treatment of cancer, such as the agents identified above.

It will be understood by one of ordinary skill in the art that the compositions and methods described herein may be adapted and modified as is appropriate for the application being addressed and that the compositions and methods described herein may be employed in other suitable applications, and that such other additions and modifications will not depart from the scope hereof. For example, in addition to the therapeutic uses described herein, the compounds and compositions of this disclosure can be used as research tools or chemical probes to, for example, understand normal cell or cancer cell biological processes, including but not limited to IRES-mediated protein synthesis, synergistic effects with mTOR inhibitors, cell division, cell proliferation, and the types of cells that are resistant or sensitive to the compounds or compositions of this disclosure. The disclosure contemplates all uses of the compounds and compositions of the disclosure, including their use in therapeutic methods and compositions for modulating cell division, their use in diagnostic assays and their use as research tools.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit IRES-mediated protein synthesis or mTOR may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g. solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cx-Cy”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “amide”, as used herein, refers to a group

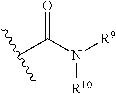

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

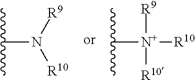

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

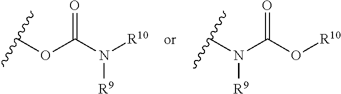

wherein R⁹ and R¹⁰ independently represent hydrogen or a hydrocarbyl group.

The term “carbocyclylalkyl”, as used herein, refers to an alkyl group substituted with a carbocycle group.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a=O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a=O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

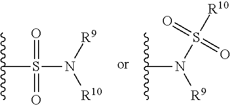

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

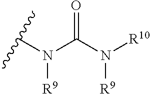

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

Abbreviations used herein include the following: mTOR, mechanistic target of rapamycin; GBM, glioblastoma; eIF4E, eukaryotic initiation factor 4E, cap-binding protein; AKT, (PKB) protein kinase B; RT-PCR, reverse transcription polymerase chain reaction; GST, glutathione-S-transferase; IRES, internal ribosome entry site; ITAF, IRES-trans-acting factor; hnRNP A1, heterogeneous nuclear ribonucleoprotein A1; EGFR, epidermal growth factor receptor; EGFRvIII, epidermal growth factor receptor variant III; PTEN, phosphatase and tensin homolog; PI3K; phosphoinositide 3-kinase; SAPK2/p38, stress-activated protein kinase 2; ECMV, encephalomyocarditis virus; RRM, RNA recognition motif; ANOVA, analysis of variance.

### Discussion

The present disclosure shows that the IRES inhibitors disclosed herein display strong synergistic anti-GBM activities when combined with mTOR kinase inhibitors. The present disclosure identifies improved IRES inhibitors, including IRES-J007, which are theorized to target the ITAF, hnRNP A1. IRES-J007 binds to a small pocket structure within the RRM-containing fragment of hnRNP A1, UP1. The pocket is within close proximity to RRM2 and inhibitor binding to hnRNP A1 blocked the ITAFs ability to associate with either the cyclin D1 or c-MYC IRESs. Furthermore, there is a synergistic antiproliferative effect of these compounds when used in combination with PP242 in vitro and in xenografted GBM cells in mice. Finally, the mRNA translational state of the cyclin D1 and c-MYC mRNAs is markedly reduced in vivo following cotherapy with PP242.

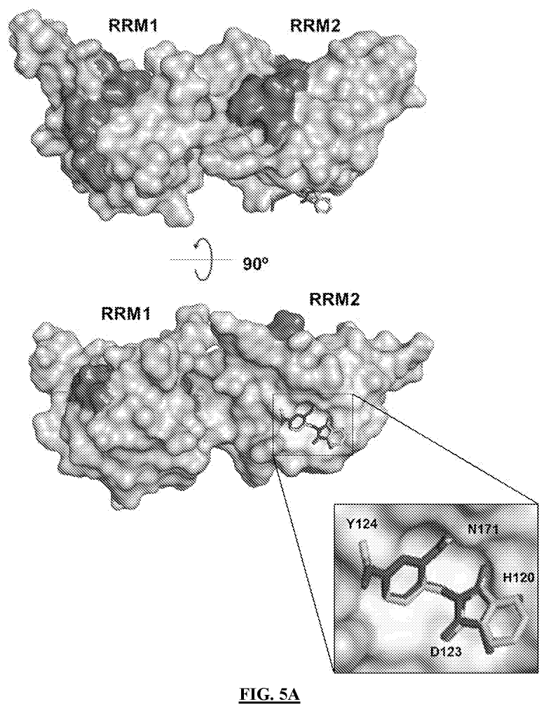

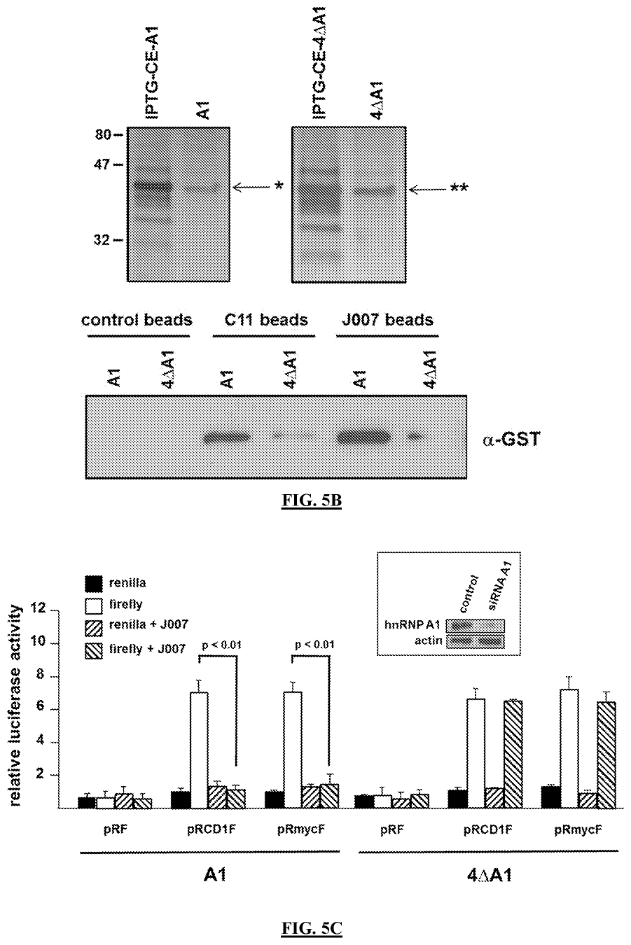

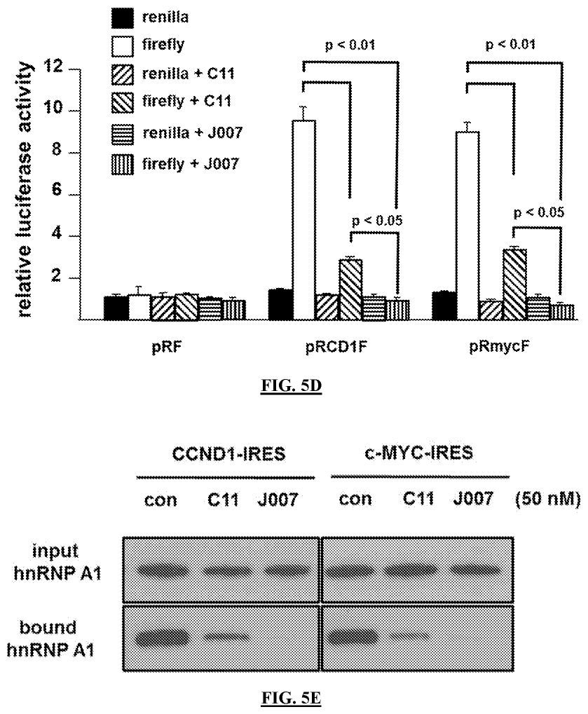

These inhibitors appear to disrupt hnRNP A1-cyclin D1 or -c-MYC IRES binding by binding a small pocket within close proximity to RRM2 altering the conformation of the ITAF to preclude IRES interaction. The improved ability of IRES-J007 to block cyclin D1 or c-MYC IRES activity may be due to IRES-J007's ability to stabilize a conformation of hnRNP A1 which binds IRES RNA less effectively. This is supported by experiments shown in FIGS. 5C and 5D, in which IRESJ007 appears to more efficiently block IRES activity and IRES RNA binding relative to the parent inhibitor. While hnRNP A1-mediated it cannot be ruled out that additional properties of hnRNP A1 which may be affected by inhibitor binding could contribute to its synergistic anti-GBM effects in combination with mTOR inhibition. As hnRNP A1 is a nuclear-cytoplasmic shuttling protein (31-34) these inhibitors may have additional effects on the cellular distribution of hnRNP A1 which contributes to their ability to block IRES activity, although in initial experiments with C11 significant nuclear redistribution was not observed in cells following exposure. The inhibitor docking studies discussed below suggest that compounds of Formula I bind to a small pocket within close proximity to RRM2. The residues in this pocket are well conserved between species and the pocket appears to have a unique surface structure. This pocket structure was superimposed on other known binding pocket structures to identify structural similarities; no similar pockets were identified in the Multiple-sketches (PoSSuM and ProBiS databases). This suggests that this surface is distinct and that the C11 and IRES-J007 inhibitors may be less likely to exhibit off-target effects.

Recent crystallographic studies with UP1 bound to the HIV exon splicing silencer 3 stem loop (SL3ESS3) suggest that UP1 binds this RNA via a short three nucleotide loop recognition element (35). The structure revealed that RRM1 and inter-RRM linker fold to form a pocket that sequesters the RNA while RRM2 does not interact with the RNA. Mutagenesis experiments of conserved salt-bridge interactions located on the opposite side of the RNA binding surface suggests RRM1 and RRM2 are conformationally coupled. If UP1 interacts with the cyclin D1 or c-MYC IRESs in a similar manner, it is conceivable that binding of C₁₁ or IRES-J007 near RRM2 may have widespread conformational effects on RRM1 of UP1 as to inhibit binding to the IRES RNAs.

This disclosure will be better understood from the Experimental Details which follow. However, one skilled in the art will readily appreciate that the specific methods and results discussed are merely illustrative of the disclosure as described more fully in the embodiments which follow thereafter.

### EXAMPLES

### Example 1: Synthesis of Disclosed Compounds

### General Synthetic Procedures

All reactions were carried out under open-air condition unless otherwise specified. Chemicals and solvents were used as received (mostly purchased from Sigma-Aldrich, Alfa Aesar, or TCI in ≥95% purity), some solvents or reagents were purified according to literature procedures if necessary. ¹H NMR spectra were recorded on a Bruker spectrometer at 500 MHz and are reported relative to deuterated solvent signals (CDCl₃ δ 7.26; DMSO-d₆ δ 2.48 ppm). Data for ¹H NMR spectra are reported as follows: chemical shift (ppm, 6), multiplicity, coupling constant (Hz) and integration. Splitting patterns are designated as follows: s, singlet; d, doublet; dd, doublet of doublets; t, triplet; td, triplet of doublets; m, multiplet. ¹³C NMR spectra were recorded on a Bruker spectrometer at 125 MHz and are reported relative to deuterated solvent signals (CHCl₃ δ 77.0; DMSO-d₆ δ 40.0 ppm). ¹⁹F NMR spectra were recorded on a Bruker spectrometer at 376.3 MHz and are reported relative to external Freon-113 in benzene (δ −73.75 ppm). The chemical shift data for ¹³C and ¹⁹F NMR spectra are reported in parts per million (ppm, 6). Melting points were obtained using Buchi B-545 melting point apparatus and are uncorrected. The reactions were monitored with a silica gel TLC plate under UV light (254 and 365 nm) followed by visualization with a ninhydrin or phospho-molybdic acid staining solution. Column chromatography was performed on silica gel 60, 230-400 mesh. DART-HRMS spectra were collected on a Thermo Exactive Plus MSD (Thermo Scientific) equipped with an ID-CUBE ion source and a Vapur Interface (IonSense). Both the source and MSD were controlled by Excalibur, version 3.0. The purity of the compounds was assayed by high field proton and carbon NMR and was ≥95%.

### Representative Procedure for Syntheses of IRES-J000, J001, J002 and J007.¹

1-((2,4-Dimethoxyphenyl)methyl)-1H-pyrrole-2,5-dione (IRES-J000). To an acetic acid (10 mL) solution of maleic anhydride (118 mg, 1.2 mmol, 1.2 eq) was added 2,4-dimethoxybenzylamine (0.15 mL, 1.0 mmol, 1.0 eq) at room temperature. The reaction mixture was refluxed for 24 h until the starting material was completely consumed and then the mixture was concentrated in vacuo. The residue was diluted with ethyl acetate (80 mL) and washed with water (2×20 mL) and brine (20 mL). The organic layer was dried with MgSO₄, filtered and concentrated in vacuo. The residue was purified by flash column chromatography over silica gel (hexane/ethyl acetate, 10:1, v/v) to afford the desired product IRES-J000 (43 mg, 15%) as a pale yellow solid: Rf=0.5 (hexane/ethyl acetate, 2:1, v/v); mp 69-71° C.; ¹H NMR (CDCl₃, 500 MHz) δ7.10 (d, J=9.0 Hz, 1H), 6.68 (s, 2H), 6.42 (s, 1H), 6.41 (dd, J=7.5, 2.5 Hz, 1H), 4.65 (s, 2H), 3.79 (s, 3H), 3.77 (s, 3H) ppm; ¹³C NMR (CDCl₃, 125 MHz) δ 170.6, 160.6, 158.2, 134.1, 130.0, 116.6, 103.9, 98.5, 55.42, 55.37, 36.5 ppm; HRMS-ESI (m/z): [M+H]⁺ calcd for C₁₃H₁₃NO₄ 248.09228; found, 248.09164.

1-Phenylmethyl-1H-pyrrole-2,5-dione (IRES-J001). Yellow solid (27% yield): Rf=0.4 (hexane/ethyl acetate, 5:1, v/v); mp 70-72° C.; ¹H NMR (DMSO-d₆, 500 MHz) δ 7.31 (t, J=7.5 Hz, 2H), 7.24 (t, J=7.5 Hz, 1H), 7.20 (d, J=7.0 Hz, 2H), 7.06 (s, 2H), 4.57 (s, 2H) ppm; ¹³C NMR (DMSO-d₆, 125 MHz) δ 171.3, 137.2, 135.2, 129.0, 127.9, 127.7, 40.9 ppm; HRMS-ESI (m/z): [M+H]⁺ calcd for C₁₁H₁₀NO₂ 188.07115; found, 188.0705. These data are in agreement with those previously reported.

1-((4-Methoxyphenyl)methyl)-1H-pyrrole-2,5-dione (IRES-J002). White powder (46% yield): Rf=0.15 (hexane/ethyl acetate, 5:1, v/v); mp 105-106° C.; ¹H NMR (DMSO-d₆, 500 MHz) δ 7.14 (d, J=8.5 Hz, 2H), 7.03 (s, 2H), 6.85 (d, J=8.5 Hz, 2H), 4.49 (s, 2H), 3.70 (s, 3H) ppm; ¹³C NMR (DMSO-d₆, 125 MHz) δ 171.3, 159.1, 135.1, 129.3, 129.2, 114.4, 55.5, 40.5 ppm; HRMS-ESI (m/z): [M+H]⁺ calcd for C₁₂H₁₂NO₃ 218.08172; found, 218.08125. These data are in agreement with those previously reported.

2-((2,4-Dimethoxyphenyl)methyl)isoindoline-1,3-dione (IRES-J007). White solid (84% yield): Rf=0.3 (hexane/ethyl acetate, 5:1, v/v); mp 149-151° C.; ¹H NMR (DMSO-d₆, 500 MHz) δ 7.89-7.85 (m, 2H), 7.85-7.52 (m, 2H), 6.95 (d, J=8.5 Hz, 1H), 6.54 (d, J=2.5 Hz, 1H), 6.41 (dd, J=8.5, 2.5 Hz, 1H), 4.64 (s, 2H), 3.76, (s, 3H), 3.70 (s, 3H) ppm; ¹³C NMR (DMSO-d₆, 125 MHz) δ 168.2, 160.4, 157.9, 135.0, 132.1, 128.8, 123.6, 116.6, 105.0, 98.8, 56.0, 55.7, 36.5 ppm; HRMS-ESI (m/z): [M+H]⁺ calcd for C₁₇H₁₆NO₄ 298.10793; found, 298.10669.

### Representative Procedure for Syntheses of IRES-J003, J006 and J008.

1-((4-Fluorophenyl)methyl)-1H-pyrrole-2,5-dione (IRES-J003). To a solution of maleic anhydride (98 mg, 1.0 mmol, 1.0 eq) in tetrahydrofuran (10 mL) was added 4-fluorobenzylamine (0.12 mL, 1.0 mmol, 1.0 eq) at room temperature and the mixture was refluxed for 3 h. After evaporation of the excess solvent, the residue was dissolved in acetic anhydride (5 mL) and sodium acetate (16 mg, 0.2 mmol, 0.2 eq) was added to the mixture. The reaction mixture was refluxed for 3 h and then concentrated in vacuo. The residue was diluted with ethyl acetate (80 mL) and washed with water (2×20 mL) and brine (20 mL). The organic layer was dried with MgSO₄, filtered and concentrated in vacuo. The residue was purified by flash column chromatography over silica gel (hexane/ethyl acetate, 10:1, v/v) to afford the desired product IRES-J003 (80 mg, 39%) as white solid: Rf=0.5 (hexane/ethyl acetate, 3:1, v/v); mp 93-95° C.; ¹H NMR (CDCl₃, 500 MHz) δ 7.32 (dd, J=7.5, 4.5 Hz, 2H), 7.00 (t, J=8.5 Hz, 2H), 6.70 (s, 2H), 4.63 (s, 2H) ppm; ¹³C NMR (DMSO, 125 MHz) δ 170.3, 162.4 (d, J=245.0 Hz, 1C), 134.3, 132.0 (d, J=3.25 Hz, 1C), 130.4 (d, J=8.1 Hz, 1C), 115.6 (d, J=21.4 Hz, 1C), 40.7 ppm; ¹⁹F NMR (CDCl₃, 376 MHz, ¹H decoupled) δ −114.20 ppm; HRMS-ESI (m/z): [M+H]⁺ calcd for C₁₁H₉FNO₂ 206.06173; found, 206.0611. These data are in agreement with those previously reported.

1-((2,4-Dimethoxyphenyl)methyl)pyrrolidine-2,5-dione (IRES-J006). Pale yellow solid (49% yield): Rf=0.5 (hexane/ethyl acetate, 2:1, v/v); mp 80-82° C.; ¹H NMR (DMSO-d₆, 500 MHz) δ 7.85 (d, J=8.5 Hz, 1H), 6.53 (d, J=2.5 Hz, 1H), 6.40 (dd, J=8.5, 2.5 Hz, 1H), 4.40 (s, 2H), 3.76 (s, 3H), 3.71 (s, 3H), 2.67 (s, 4H) ppm; ¹³C NMR (DMSO-d₆, 125 MHz) δ 178.0, 160.3, 157.9, 128.2, 116.1, 104.9, 98.7, 56.0, 55.7, 36.6, 28.6 ppm; HRMS-ESI (m/z): [M+H]⁺ calcd for C₁₃H₁₆NO₄ 250.10793; found, 250.10706. These data are in agreement with those previously reported.

1-(4-Methoxyphenyl)-1H-pyrrole-2,5-dione (IRES-J008). Yellow solid (65% yield): Rf=0.2 (hexane/ethyl acetate, 3:1, v/v); mp 152-155° C.; ¹H NMR (DMSO-d₆, 500 MHz) δ 7.21 (d, J=9.0 Hz, 2H), 7.13 (s, 2H), 7.01 (d, J=9.0 Hz, 2H), 3.77 (s, 3H) ppm; ¹³C NMR (DMSO-d₆, 125 MHz) δ 170.7, 159.1, 135.1, 128.8, 124.5, 114.6, 55.8 ppm; HRMS-ESI (m/z): [M]⁺ calcd for C₁₁H₉NO₃ 203.05824; found, 203.05650. These data are in agreement with those previously reported.

### Representative Procedure for Syntheses of IRES-J004 and J005

1-((2,4-Dimethoxyphenyl)methyl)-3,4-dimethyl-1H-pyrrole-2,5-dione (IRES-J004). To a solution of 2,3-dimethylmaleic anhydride (126 mg, 1.0 mmol, 1.0 eq) in tetrahydrofuran (10 mL) was added 2,4-dimethoxybenzylamine (0.15 mL, 1.0 mmol, 1.0 eq) at 0° C. The reaction mixture was refluxed for 2 h until the starting material was completely consumed and then concentrated in vacuo. The residue was purified by flash column chromatography over silica gel (hexane/ethyl acetate, 10:1, v/v) to afford the desired product IRES-J004 (153 mg, 56%) as a white powder: Rf=0.3 (hexane/ethyl acetate, 5:1, v/v); mp 105-107° C.; ¹H NMR (CDCl₃, 500 MHz) δ 7.08 (d, J=8.0 Hz, 1H), 6.41 (bs, 1H), 6.40 (dd, J=8.0, 2.0 Hz, 1H), 4.62 (s, 2H), 3.80 (s, 3H), 3.77 (s, 3H), 1.95 (s, 6H) ppm; ¹³C NMR (CDCl₃, 125 MHz) δ 172.0, 160.4, 158.1, 137.1, 129.8, 117.2, 103.9, 98.5, 55.5, 55.4, 36.3, 8.7 ppm; HRMS-ESI (m/z): [M+H]⁺ calcd for C₁₅H₁₈NO₄ 276.12358; found, 276.12216.

1-((2,4-Dimethoxyphenyl)methyl)-3,4-diphenyl-1H-pyrrole-2,5-dione (IRES-J005). Yellow solid (40% yield): Rf=0.4 (hexane/ethyl acetate, 5:1, v/v); mp 97-99° C.; NMR (CDCl₃, 500 MHz) δ 7.48-7.46 (m, 4H), 7.38-7.34 (m, 6H), 7.25 (d, J=9.0 Hz, 1H), 6.45 (s, 1H), 6.44 (dd, J=9.0, 2.5 Hz, 1H), 4.80 (s, 2H), 3.83 (s, 3H), 3.78 (s, 3H) ppm; ¹³C NMR (CDCl₃, 125 MHz) δ 170.5, 160.6, 158.3, 136.1, 130.5, 130.0, 129.7, 128.8, 128.5, 116.9, 104.0, 98.5, 55.5, 55.4, 36.8 ppm; HRMS-ESI (m/z): [M+H]⁺ calcd for C₂₅H₂₂NO₄ 400.15488; found, 400.15234.

1-Methyl-3-(phenylmethyl)pyrimidine-2,4(1H,3H)-dione (IRES-J009). To a suspension of uracil (1.12 g, 10.0 mmol, 1.0 eq) in 1,2-dichloroethane (20 mL), were added hexamethyldisilazane (8.4 mL, 40.0 mmol, 4.0 eq) and chlorotrimethylsilane (0.67 mL, 5.3 mmol, 0.53 eq) and the mixture refluxed for 4 h. The resulting mixture was cooled to room temperature, the solvent was removed under reduced pressure and 1,2-dichloroethane (10 mL) was added. To the reaction mixture were added iodomethane (2.49 mL, 40.0 mmol, 4.0 eq) and iodine (25.4 mg, 0.1 mmol, 0.01 eq), and the mixture was refluxed for 24 h. The excess solvent was removed in vacuo and the residue was purified by flash column chromatography over silica gel (dichloromethane/methanol, 30:1, v/v) to afford the desired product (1-methylpyrimidine-2,4(1H,3H)-dione, 645 mg, 51%) as brown solid: Rf=0.5 (dichloromethane/methanol, 10:1, v/v); ¹HNMR (DMSO-d₆, 500 MHz) δ 11.20 (s, 1H), 7.59 (d, J=7.5 Hz, 1H), 5.49 (d, J=8.0 Hz, 1H), 3.20 (s, 3H) ppm; ¹³C NMR (DMSO-d₆, 125 MHz) δ 164.4, 151.7, 146.9, 101.0, 35.7 ppm. These data are in agreement with those previously reported.

To a solution of 1-methylpyrimidine-2,4(1H,3H)-dione (63 mg, 0.5 mmol, 1.0 eq) in ethanol (5 mL) was added sodium hydroxide (40 mg, 1.0 mmol, 2.0 eq) and benzyl bromide (0.12 mL, 1.0 mmol, 2.0 eq) at room temperature. The mixture was stirred for 72 h at room temperature and then concentrated in vacuo. The residue was diluted with ethyl acetate (80 mL) and washed with water (2×20 mL) and brine (20 mL). The organic layer was dried with MgSO₄, filtered and concentrated in vacuo. The residue was purified by flash column chromatography over silica gel (hexane/ethyl acetate, 3:1, v/v) to afford the desired product IRES-J009 (60 mg, 56%) as white solid: mp 108-110° C.; ¹H NMR (DMSO-d₆, 500 MHz) δ 7.69 (d, J=7.5 Hz, 1H), 7.29-7.22 (m, 5H), 5.69 (d, J=7.5 Hz, 1H), 4.95 (s, 2H), 3.27 (s, 3H) ppm; ¹³C NMR (DMSO-d₆, 125 MHz) δ 163.1, 151.9, 145.7, 137.7, 128.8, 128.1, 127.6, 100.2, 43.8, 36.9 ppm; HRMS-ESI (m/z): [M+H]⁺ calcd for C₁₂H₁₃N₂O₂ 217.09770; found, 217.09686.

### Representative Procedure for Syntheses of IRES-J010 and J013-J015.

2-(Phenylmethyl)isoindoline-1,3-dione (IRES-J010). To a solution of phthalic anhydride (444 mg, 3.0 mmol, 1.0 eq) in toluene (15 mL) was added benzylamine (0.36 mL, 3.3 mmol, 1.1 eq) at room temperature. The reaction mixture was refluxed for 5 h until the starting material was completely consumed and then the mixture was concentrated in vacuo. The residue was purified by flash column chromatography over silica gel (hexane/ethyl acetate, 5:1, v/v) to afford the desired product IRES-J010 (565 mg, 79%) as white powder: Rf=0.6 (hexane/ethyl acetate, 5:1, v/v); mp 108-110° C.; ¹H NMR (DMSO-d₆, 500 MHz) δ 7.89-7.86 (m, 2H), 7.85-7.83 (m, 2H), 7.33-7.24 (m, 5H), 4.75 (s, 2H) ppm; NMR (DMSO-d₆, 125 MHz) δ 168.2, 137.1, 135.1, 132.0, 129.1, 127.9, 127.8, 123.7, 41.3 ppm; HRMS-ESI (m/z): [M+H]⁺ calcd for C₁₂H₁₂NO₂ 238.08680; found, 238.08607. These data are in agreement with those previously reported.

2-(4-Methoxyphenyl)isoindoline-1,3-dione (IRES-J013). Yellow solid (73% yield): Rf=0.2 (hexane/ethyl acetate, 5:1, v/v); mp 157-159° C.; ¹H NMR (DMSO-d₆, 500 MHz) δ 7.94-7.93 (m, 2H), 7.89-7.87 (m, 2H), 7.33 (d, J=9.0 Hz, 2H), 7.05 (d, J=9.0 Hz, 2H), 3.79 (s, 3H) ppm; ¹³C NMR (DMSO-d₆, 125 MHz) δ 167.8, 159.3, 135.1, 132.1, 129.3, 124.9, 123.8, 114.6, 55.9 ppm; HRMS-ESI (m/z): [M+H]⁺ calcd for C₁₅H₁₂₇NO₃ 254.08172; found, 254.08066. These data are in agreement with those previously reported.

2-(4-(Dimethylamino)phenyl)isoindoline-1,3-dione (IRES-J014). Yellow solid (73% yield): Rf=0.4 (hexane/ethyl acetate, 5:1, v/v); mp 264-267° C.; ¹HNMR (DMSO-d₆, 500 MHz) δ 7.92-7.90 (m, 2H), 7.87-7.86 (m, 2H), 7.18 (d, J=8.5 Hz, 2H), 6.78 (d, J=9.0 Hz, 2H), 2.93 (s, 6H) ppm; ¹³C NMR (DMSO-d₆, 125 MHz) δ 168.0, 150.5, 135.0, 132.1, 128.6, 123.7, 120.6, 112.5, 40.6 ppm HRMS-ESI (m/z): [M+H]⁺ calcd for C₁₆H₁₅N₂O₂ 267.11335; found, 267.11206. These data are in agreement with those previously reported.

2-(3-Methoxypropyl)isoindoline-1,3-dione (IRES-J015). White solid (68% yield): Rf=0.4 (hexane/ethyl acetate, 5:1, v/v); mp 50-52° C.; ¹HNMR (DMSO-d₆, 500 MHz) δ 7.86-7.83 (m, 2H), 7.82-7.80 (m, 2H), 3.61 (t, J=7.0 Hz, 2H), 3.32 (t, J=6.0 Hz, 2H), 3.16 (s, 3H), 1.79 (tt, J=6.5, 6.0 Hz, 2H) ppm; ¹³C NMR (DMSO-d₆, 125 MHz) δ 168.4, 134.8, 132.2, 123.4, 70.0, 58.4, 35.6, 28.5 ppm; HRMS-ESI (m/z): [M+H]⁺ calcd for C₁₂H₁₄NO₃ 220.09737; found, 220.09663. These data are in agreement with those previously reported.

2-(Phenylamino)isoindoline-1,3-dione (IRES-J011). To a solution of N-hydroxyphthalimide (489 mg, 3.0 mmol, 1.0 eq) in pH 7.0 phosphate buffer (30 mL) was added phenylhydrazine (0.293 mL, 3.0 mmol, 1.0 eq) at room temperature. The mixture was stirred for 15 h at room temperature and then the mixture was filtered and washed with water. The filtered solid was diluted with ethyl acetate (150 mL) and washed with 5% aq. HCl (30 mL×2) and the organic phase was dried with brine and MgSO₄, filtered and concentrated in vacuo. No further purification was needed to afford the desired product IRES-J011 (620 mg, 87%) as a bright yellow solid: Rf=0.5 (hexane/ethyl acetate, 2:1, v/v); mp 182-184° C.; ¹HNMR (DMSO-d₆, 500 MHz) δ 8.54 (s, 1H), 7.94-7.92 (m, 2H), 7.91-7.89 (m, 2H), 7.15 (t, J=7.5 Hz, 2H), 6.77 (t, J=7.5 Hz, 1H), 6.71 (d, J=7.5 Hz, 2H) ppm; ¹³C NMR (DMSO-d₆, 125 MHz) δ 167.1, 147.3, 135.5, 130.1, 129.5, 124.0, 120.2, 112.6 ppm; HRMS-ESI (m/z): [M+H]⁺ calcd for C₁₄H₁₁N₂O₂ 239.08205; found, 239.08113. These data are in agreement with those previously reported.

2-(Diphenylamino)isoindoline-1,3-dione (IRES-J012). To a solution of N-hydroxyphthalimide (489 mg, 3.0 mmol, 1.0 eq) in pH 7.0 phosphate buffer (30 mL) was added N,N-diphenyl-hydrazine HCl (662 mg, 3.0 mmol, 1.0 eq) at room temperature. The mixture was refluxed for 3 h and then the mixture was filtered and washed with water. The filtered solid was diluted with ethyl acetate (150 mL) and washed with 5% aq. HCl (50 mL×3) and the organic phase was dried with brine and MgSO₄, filtered and concentrated in vacuo. No further purification was needed to afford the desired product IRES-J012 (200 mg, 21%) as a pale green solid: Rf=0.4 (hexane/ethyl acetate, 5:1, v/v); mp 159-161° C.; ¹HNMR (DMSO-d₆, 500 MHz) δ 7.97-7.92 (m, 4H), 7.31 (t, J=8.0 Hz, 4H), 7.10 (d, J=8.0 Hz, 4H), 7.06 (t, J=7.5 Hz, 2H) ppm; ¹³C NMR (DMSO-d₆, 125 MHz) δ 166.5, 144.4, 135.9, 130.0, 129.6, 124.4, 124.1, 119.7 ppm; HRMS-ESI (m/z): [M]⁺ calcd for C₂₀H₁₄N₂O₂ 314.10553; found, 314.10420. These data are in agreement with those previously reported.

2-(Phenylmethyl)isoquinoline-1,3(2H,4H)-dione (IRES-J016). To a solution of homophthalic anhydride (162 mg, 1.0 mmol, 1.0 eq) in toluene (10 mL) was added benzylamine (0.12 mL, 1.1 mmol, 1.1 eq) at room temperature. The reaction mixture was refluxed for 3 h then cooled to room temperature. The resulting solid (unreacted homophthalic anhydride) was filtered and washed with ethyl acetate/hexane mixture, and the organic phase was concentrated in vacuo. The residue was purified by flash column chromatography over silica gel (hexane/ethyl acetate, 3:1, v/v) to afford the desired product IRES-J012 (53 mg, 21%) as a yellow solid: Rf=0.2 (hexane/ethyl acetate, 3:1, v/v); mp 124-126° C.; ¹H NMR (CDCl₃, 500 MHz) δ 8.21 (d, J=8.0 Hz, 1H), 7.57 (t, J=7.5 Hz, 1H), 7.46 (d, J=7.5 Hz, 2H), 7.43 (t, J=7.5 Hz, 1H), 7.29 (t, J=7.5 Hz, 2H), 7.26-7.22 (m, 2H), 5.18 (s, 2H), 4.06 (s, 2H) ppm; ¹³C NMR (CDCl₃, 125 MHz) δ 169.9, 164.9, 137.1, 134.1, 133.7, 129.3, 129.0, 128.4, 127.8, 127.6, 127.1, 125.4, 43.3, 36.5 ppm; HRMS-ESI (m/z): [M+H]⁺ calcd for C₁₆H₁₄NO₂ 252.10245; found, 252.10184. These data are in agreement with those previously reported.

3-(Phenylmethyl)quinazoline-2,4(1H,3H)-dione (IRES-J017). A mixture of anthranilic acid (822 mg, 6.0 mmol, 1.0 eq) and triphosgene (605 mg, 2.04 mmol, 0.34 eq) in dry tetrahydrofuran (30 mL) was stirred for 4 h at 40-50° C. under an argon atmosphere. The mixture was concentrated in vacuo and the resulting solid was filtered and washed with hexane. No further purification was needed to afford the desired product (2H-benzo[d][1,3]oxazine-2,4(1H)-dione, 930 mg, 95%) as a dark green solid: ¹H NMR (DMSO-d₆, 500 MHz) δ 11.71 (s, 1H), 7.89 (d, J=7.5 Hz, 1H), 7.72 (t, J=7.5 Hz, 1H), 7.23 (t, J=7.5 Hz, 1H), 7.13 (d, J=8.0 Hz, 1H) ppm; ¹³C NMR (DMSO-d₆, 125 MHz) δ 160.4, 147.6, 141.9, 137.4, 129.4, 124.0, 115.8, 110.8 ppm. These data are in agreement with those previously reported.

To a dimethylacetamide (2 mL) solution of 2H-benzo[d][1,3]oxazine-2,4(1H)-dione (408 mg, 2.5 mmol, 1.0 eq) and urea (150 mg, 2.5 mmol, 1.0 eq) was added benzylamine (0.33 mL, 3.0 mmol, 1.2 eq) at room temperature. The reaction mixture was irradiated in a microwave reactor at 250 W for 5 min at 160° C. with vigorous stirring. The reaction mixture was cooled to room temperature and added water (3 mL) and then the resulting solid was filtered and washed with methanol and hexane. No further purification was needed to afford the desired product IRES-J017 (405 mg, 64%) as a white solid: Rf=0.3 (hexane/ethyl acetate, 2:1, v/v); mp 228-229° C.; ¹H NMR (CDCl₃, 500 MHz) δ 9.42 (s, 1H), 8.14 (d, J=8.0 Hz, 1H), 7.60 (t, J=7.5 Hz, 1H), 7.52 (d, J=7.5 Hz, 2H), 7.32-7.29 (m, 2H), 7.26-7.21 (m, 2H), 7.03 (d, J=8.0 Hz, 1H), 5.27 (s, 2H) ppm; ¹³C NMR (CDCl₃, 125 MHz) δ 162.3, 151.6, 138.4, 136.8, 135.1, 128.9, 128.7, 128.5, 127.7, 123.5, 114.8, 114.7, 44.2 ppm; HRMS-ESI (m/z): [M+H]⁺ calcd for C₁₅H₁₃N₂O₂ 253.09770; found, 253.09610. These data are in agreement with those previously reported.

1-Methyl-3-(phenylmethyl)quinazoline-2,4(1H,3H)-dione (IRES-J018). To a solution of 3-benzylquinazoline-2,4(1H,3H)-dione (IRES-J017, 126 mg, 0.5 mmol, 1.0 eq) and potassium carbonate (207 mg, 1.5 mmol, 3.0 eq) in dimethylformamide (5 mL) was added iodomethane (0.15 mL, 2.5 mmol, 5.0 eq) at 0° C. The reaction mixture was warmed to room temperature and stirred for 3 h, and then concentrated in vacuo. The residue was diluted with ethyl acetate (80 mL) and washed with water (2×20 mL) and brine (20 mL). The organic layer was dried with MgSO₄, filtered and concentrated in vacuo. The residue was purified by flash column chromatography over silica gel (hexane/ethyl acetate, 2:1, v/v) to afford the desired product IRES-J018 (120 mg, 90%) as a white powder: mp 131-133° C.; ¹H NMR (DMSO-d₆, 500 MHz) δ 8.05 (dd, J=8.0, 1.5 Hz, 1H), 7.78 (td, J=8.5, 1.5 Hz, 1H), 7.45 (d, J=8.5 Hz, 1H), 7.32-7.22 (m, 6H), 5.12 (s, 2H), 3.51 (s, 3H) ppm; NMR (DMSO-d₆, 125 MHz) δ 161.6, 150.9, 141.0, 137.7, 136.0, 128.8, 128.3, 128.1, 127.6, 123.3, 115.23, 115.17, 44.7, 31.2 ppm; HRMS-ESI (m/z): [M+H]⁺ calcd for C₁₆H₁₅N₂O₂ 267.11335; found, 267.11230. These data are in agreement with those previously reported.

2-Phenyl-2,3-dihydrophthalazine-1,4-dione (IRES-J019). To a solution of phthalic anhydride (444 mg, 3.0 mmol, 1.0 eq) in 10% aq. HCl (30 mL) was added phenylhydrazine (0.35 mL, 3.6 mmol, 1.2 eq) dropwise and the reaction mixture was refluxed for 15 h. The resulting solid in reaction mixture was filtered off and washed with water and dried in vacuo. No further purification was needed to afford the desired product IRES-J019 (665 mg, 93%) as a pale peach solid: Rf=0.25 (hexane/ethyl acetate, 2:1, v/v); mp 216-218° C.; ¹H NMR (DMSO-d₆, 500 MHz) δ 11.82 (s, 1H), 8.28 (dd, J=7.5, 1.0 Hz, 1H), 8.00 (dd, J=7.5, 1.0, 1H), 7.95 (td, J=7.5, 1.0 Hz, 1H), 7.90 (td, J=7.5, 1.0 Hz, 1H), 7.62 (d, J=7.0 Hz, 2H), 7.47 (t, J=7.5 Hz, 2H), 7.35 (t, J=7.5 Hz, 1H) ppm; ¹³C NMR (DMSO-d₆, 125 MHz) δ 157.8, 150.9, 142.2, 134.1, 133.0, 129.7, 128.9, 127.6, 127.3, 126.4, 125.1, 124.7 ppm; HRMS-ESI (m/z): [M+H]⁺ calcd for C₁₄H₁₁N₂O₂ 239.08205; found, 239.08102. These data are in agreement with those previously reported.

2-Methyl-3-phenyl-2,3-dihydrophthalazine-1,4-dione (IRES-J020). To a solution of 2-phenyl-2,3-dihydrophthalazine-1,4-dione (IRES-J019, 119 mg, 0.5 mmol, 1.0 eq) and potassium carbonate (207 mg, 1.5 mmol, 3.0 eq) in dimethylformamide (5 mL) was added iodomethane (0.15 mL, 2.5 mmol, 5.0 eq) at 0° C. The reaction mixture was warmed to room temperature and stirred for 4 h, and then concentrated in vacuo. The residue was diluted with ethyl acetate (80 mL) and washed with water (2×20 mL) and brine (20 mL). The organic layer was dried with MgSO₄, filtered and concentrated in vacuo. The residue was purified by flash column chromatography over silica gel (hexane/ethyl acetate, 2:1, v/v) to afford the desired product IRES-J020 (80 mg, 63%) as a pale yellow powder: mp 115-117° C.; ^{1H} NMR (DMSO-d₆, 500 MHz) δ 8.31 (d, J=8.0 Hz, 1H), 8.01 (dd, J=7.5, 1.5 Hz, 1H), 7.97 (td, J=8.0, 1.5 Hz, 1H), 7.93 (td, J=7.5, 1.5 Hz, 1H), 7.70 (d, J=7.5 Hz, 2H), 7.48 (t, J=7.5 Hz, 2H), 7.36 (t, J=7.5 Hz, 1H), 3.96 (s, 3H) ppm; ¹³C NMR (DMSO-d₆, 125 MHz) δ 157.9, 150.2, 142.3, 134.4, 133.2, 129.3, 128.9, 127.6, 127.5, 126.0, 124.5, 123.9, 54.8 ppm; HRMS-ESI (m/z): [M+H]⁺ calcd for C₁₅H₁₃N₂O₂ 253.09770; found, 253.09662.

### Example 2: General Biological Procedures

### Cell Lines, Constructs and Transfections—

Glioblastoma lines LN229 and LN18 were obtained from ATCC (Manassas, Va.). Paul Mischel (Ludwig Institute, UCSD) kindly provided the LN428 line and the SF763 line was from the UCSF Neurosurgery Tissue Bank (UCSF). 293T cells were kindly provided by Norimoto Yanagawa (UCLA). Normal mature human neurons were obtained from ScienCell (Carlsbad, Calif.). The dicistronic constructs pRF, pRCD1F, pRmycF and pRp27F have been described previously (18). The pRECMVF construct was generously provided by Eric Jan (Department of Biochemistry, University of British Columbia). pGEX-2T/hnRNP A1 (full length hnRNP A1) and pGEX-2T/UP1 GST fusions were kindly provided by Ronald Hay (Centre for Gene Regulation and Expression, University of Dundee) and used to generate additional deletion mutants. To generate the hnRNP A1 alanine substitution mutants, the full length hnRNP A1 containing plasmid was mutagenized using the QuikChange Lightning Site-Directed Mutagenesis kit (Agilent Technologies) using appropriate mutagenic primers according to the manufacturer. All plasmids were sequenced to verify the constructs. DNA transfections were performed using Effectene transfection reagent according to the manufacturer (Qiagen).

### Recombinant Proteins, Antibodies, Reagents and C11 Structure-Activity Relationship (SAR) Analog Preparation

Recombinant native and mutant hnRNP A1 was expressed and purified from HEK293 cells using anti-glutathione Sepharose column chromatography as previously described (18). All antibodies were from Cell Signaling, except hnRNP A1 which was obtained from Abcam. PP242 and rapamycin were obtained from LC Laboratories (Woburn, Mass.). C11 (NSC-603707) was obtained from the Developmental Therapeutics Program repository at the NCI.

### Protein and RNA Analyses

Western blot and quantitative RT-PCR analyses were performed as previously described (19). For Western blotting, cells or tissues were lysed in RIPA (lysis) buffer containing protease inhibitor cocktail and phosSTOP phosphatase inhibitor cocktail (Roche) and extracts resolved by SDSPAGE. Proteins were transferred to PVDF membranes and incubated with the indicated antibodies. For IRES reporter assays, the indicated mRNA reporters were cotransfected into cells with pSVβ-galactosidase to normalize for transfection efficiency as described previously (16). Cells were harvested 18 h following transfection and Renilla, firefly and β-galactosidase activities determined using the Dual-Glo Luciferase and β-galactosidase assay systems (Promega).

For immunopurification of eIF4E and bound RNA (21), cells were lysed in buffer containing 50 mM Tris-HCl (pH 7.4), 300 mM NaCl, 0.5% NP-40, 10 mM NaF, 1 mM sodium orthovanidate, 1 mM PMSF, 1.5% aprotinin and SUPERase-IN (ThermoFisher Scientific), 0.025 units/ml, using a Dounce homogenizer at 4° C. Extracts were cleared by centrifugation and immunoprecipitated with eIF4E or control IgG. Beads were washed and obtained RNA was subjected to quantitative RTPCR. For quantitative RT-PCR, extraction of RNA was performed using Trizol (ThermoFisher). Total RNA was then quantified and integrity assessed using an Agilent 2100 Bioanalyzer (Agilent Technology). Total RNA was reverse transcribed with random primers using the RETROscript Kit from ThermoFisher. SYBR Green quantitative PCR was performed in triplicate in 96-well optical plates on an ABI Prism 7000 Sequence Detection System (Life Technologies) according to the manufacturer's instructions. Primer sequences for CCND1, c-MYC and actin are available upon request. For RNA-pull down assays (18), cytoplasmic extracts were prepared by hypotonic lysis in buffer containing 10 mM HEPES (pH 7.5), 10 mM potassium acetate, 1.5 mM magnesium acetate, 2.5 mM DTT, 0.05% NP-40, 10 mM NaF, 1 mM sodium orthovanidate, 1 mM PMSF and 1.5% aprotinin using a Dounce homogenizer. Extracts were precleared by centrifugation, and SUPERase-IN (ThermoFisher, 0.025 units/ml) and yeast tRNA (15 μg/ml) were added and applied to an equilibrated heparin-agarose column (Bio-Rad). Eluates were further cleared with 100 μl of streptavidin-Sepharose (Sigma) for 1 h at 4° C. Following centrifugation, 10 μg of in vitro transcribed biotinylated IRES RNA (mMESSAGE Machine T7 transcription kit, ThermoFisher) was added to the supernatant and incubated for 1 h at 4° C. The protein and biotinylated RNA complexes were recovered by adding 30 μl of streptavidin-Sepharose, which was incubated for 2 h at 4° C. The complexes were washed five times in binding buffer (10 mM HEPES (pH 7.5), 90 mM potassium phosphate, 1.5 mM magnesium acetate, 2.5 mM DTT, 0.05% NP-40, 10 mM NaF, 1 mM sodium orthovanidate, 1 mM PMSF and 1.5% aprotinin) and then boiled in SDS and resolved by gel electrophoresis.

Max exon 5 alternative splicing analysis was performed as described (22). Alternative splicing was assayed using quantitative RT-PCR using primers designed to constitutive exons flanking alternative exons. Primers were: Max (85F) 5′-tcagtcccatcactccaagg-3′; MAX(85R) 5′-gcacttgacctcgccttct-3′. Reverse primers were 32P end-labeled and PCR reactions were amplified for 22 cycles and subsequently resolved by denaturing PAGE and imaged.

Polysome analyses were performed as previously described (19). Cells were lysed in buffer containing 100 μg/ml cycloheximide at 4° C. Following removal of mitochondria and nuclei, supernatants were layered onto 15-50% sucrose gradients and centrifuged at 38,000 rpm for 2 h at 4° C. in an SW40 rotor (Beckman Instruments). Gradients were fractionated into 11 1-ml fractions using a density gradient fractionator (Brandel Instruments). The profiles of the gradients were monitored at 260 nm, and RNAs from individual fractions were pooled into a nonribosomal/monosomal pool and a polysomal pool. These RNAs (100 ng) were used in real time quantitative RT-PCR analysis for the indicated transcripts.

Filter binding assays were performed as previously described (18,23). GSTtagged hnRNP A1 or hnRNP A1 deletion mutants were added to in vitro transcribed 32Plabeled RNAs corresponding to either the cyclin D1 or c-MYC IRESs in separate reactions in a volume of 10 μl in buffer containing 5 mM HEPES (pH 7.6), 30 mM KCl, 2 mM MgCl2, 200 mM DTT, 4% glycerol and 10 ng yeast tRNA for 10 min at room temperature. 8 μl of each binding reaction was applied to nitrocellulose membranes on a slot blot apparatus (Minifold II, Schleicher & Schuell). Membranes were washed and dried, and signals quantified using a phosphorimager.

### Docking Analysis of IRES Inhibitors and UP1

In silico analysis of C₁₁, IRES-J007 and UP1 was performed using AutoDock Vina (24). The steric structure of monomeric UP1 was derived from the crystal structure deposited in the RCSB PDB (1HA1). The UP1 structure was pre-processed and hydrogen atoms were added prior to docking simulation. Models were visualized using PyMOL v1.5.6 (Schrödinger, LLC).

### Photo-Cross-Linking Assays

Photo-cross-linked C11 and IRES-J007 beads were prepared as previously described (25). Activated Sepharose beads were washed three times with 1 mM aqueous HCl followed by coupling solution (100 mM NaHCO₃ and 50% dioxane mixture). A solution of photoaffinity linker in coupling solution was subsequently added to the beads and incubated at 37° C. for 2 h. After washing five times with coupling solution the beads were blocked and placed in a spin column and washed three times with water and methanol. The beads were subsequently irradiated in a UV cross-linker at 365 nm (4 J/cm2) and washed with methanol. Purified GST-tagged native or mutant hnRNP A1 proteins were added to 20 μl of C11 or J007 cross-linked or control uncross-linked beads. After incubating at 4° C. for 24 h, the beads were washed three times and bound proteins eluted in 10% SDS-PAGE sample buffer at 100° C. for 5 min. and the eluted proteins were resolved by SDS-PAGE and immunoblotted using GST antibodies.

### Cell Proliferation, Cell-Cycle Distribution and TUNEL Assays

Cells were plated into 96-well plates and after culturing for various time points, cell numbers were measured by 2,3-bis[2-methoxy-4-nitro-5-sulfophenyl]-2H-tetrazolium-5-carboxanilide inner salt (XTT) assay (Roche) as described by the manufacturer. Viability of human neurons was assessed by trypan blueexclusion. Cell-cycle analysis was done by propidium iodide staining of cells and flow cytometry as previously described (19). Cells were stained for annexin V using a FITCconjugated anti-annexin V antibody (Annexin VFITC Early Apoptosis Detection kit, Cell Signaling). TUNEL staining of tumor sections was performed using the TACSXL DAB In Situ Apoptosis Detection kit (Trevigen) according to the manufacturer's instructions (19). The combination index (CI) values were determined by using CalcuSyn v2.0 software (Biosoft) (19). Xenograft Studies-Xenografts of LN229 cells were performed in female C.B.-17-scid (Taconic) mice as previously described (14). Tumors were harvested at autopsy for Western blot analysis. Sections of paraffin-embedded tumors on slides were processed for immunohistochemistry as previously described (14,19). Statistical analysis was done with Student's t test and ANOVA models using Systat 13 (Systat Software, Chicago, Ill.). P values of less the 0.05 were considered significant.

### Example 3: C11 Inhibits Cyclin D1 and c-Myc IRES Activity in GBM Via Blockade of hnRNPA1-IRES Interactions

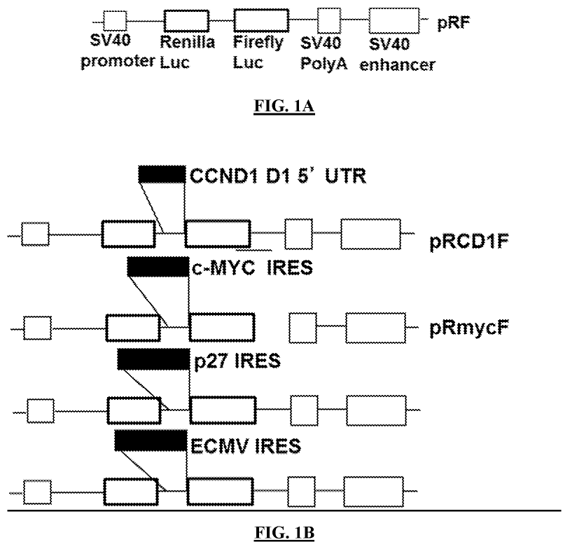

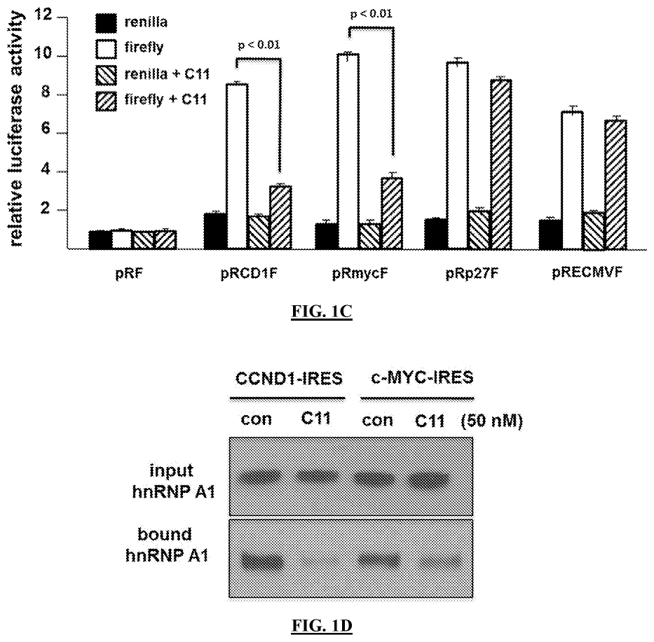

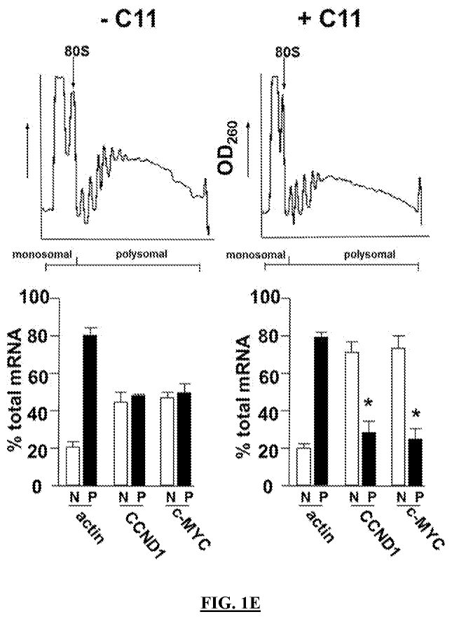

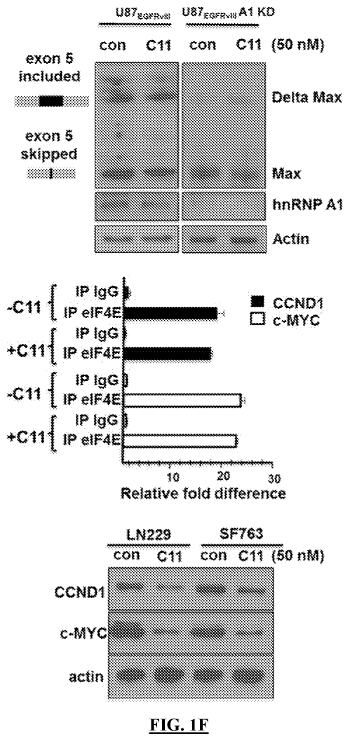

FIG. 1 shows (A) Chemical structure of C11. (B) Schematic diagrams of the dicistronic constructs used in this study. Constructs used are pRF, pRCD1F, which contains the human cyclin D1 IRES, pRmycF, containing the human c-myc IRES, pRp27F, containing the human p27Kip1 IRES and pRECMVF, containing the IRES from encephalomyocarditis virus. (C) Relative Renilla and firefly luciferase activities obtained from LN229 GBM cells transfected with the indicated constructs in the absence or presence of the inhibitor C11. The mean and +SD are shown for three independent experiments. (*, P<0.01, significantly different from firefly and firefly+C11). (D) RNA-pull down assays utilizing biotinylated cyclin D1 or c-MYC IRES RNAs. Cytoplasmic extracts of LN229 cells treated with C11 (50 nM) as indicated were incubated with biotinylated cyclin D1 or c-MYC IRES RNAs and precipitated with streptavidin-Sepharose beads. Input and bound fractions were analyzed by immunoblotting using hnRNP A1 antibodies. (E) Polysome distributions of cyclin D1, c-MYC and actin mRNAs in LN229 cells in the absence or presence of C11 (50 nM). Extracts were subjected to sucrose density centrifugation and then divided into 11 1-ml fractions, which were pooled into a nonribosomal, monosomal fraction (N, white bars) and a polysomal fraction (P, black bars). Purified RNAs were subsequently used in real time quantitative RT-PCR analysis to determine the distributions of cyclin D1, c-MYC and actin mRNAs across the gradients. Polysome tracings are shown above values obtained from the RT-PCR analyses, which are displayed graphically below. RT-PCR measurements were performed in quadruplicate and the mean and +S.D. are shown. (*, P<0.05, significantly different from CCND1 or c-MYC and CCND1+C11 or c-MYC+C11). (F) Top panel, LN229 cells were treated with C11 as indicated and RT-PCR splicing analysis for Max exon 5 performed as described in the Experimental Methods section. Middle panel, LN229 cells treated with C11 (50 nM) as indicated, were lysed and immunoprecipitated using either eIF-4E or control IgG antibodies. Bound CCND1 or c-MYC RNAs were detected via RT-PCR. The mean and +S.D. are shown for three independent experiments. Bottom panel, cyclin D1 and c-MYC protein levels from the indicated GBM cell lines in the absence or presence of C11 at 24 h following treatment. C11 specifically blocks c-Myc IRES-mediated translation initiation by disrupting the ITAF hnRNP A1-IRES interaction.

As the cyclin D1 IRES utilizes hnRNP A1 as an ITAF and its ability to initiate IRES-mediated translation initiation is regulated in a similar manner as the c-MYC IRES, it was determined whether C11 would also inhibit its IRES activity. Several dicistronic IRES mRNA reporter constructs were utilized, shown in FIG. 1B, in which the indicated IRES sequences were inserted within the intercistronic region. LN229 cells transiently transfected with these constructs were assayed for Renilla and firefly luciferase activities, which are readouts of cap-dependent and IRES-mediated translation initiation, respectively (16).

As shown in FIG. 1C, C11 significantly inhibited both cyclin D1 and c-MYC IRES activity consistent with the requirement of these IRESs for hnRNP A1 function (18). C11 however, did not affect IRES-mediated initiation from either the p27^{Kip1} or ECMV IRESs that do not utilize hnRNP A1 as an ITAF. To examine whether C11 would affect hnRNP A1 binding to the cyclin D1 or c-MYC IRESs, RNA-pull down assays were performed utilizing cell extracts from cells treated with C11. As shown in FIG. 1D, hnRNP A1 was preferentially precipitated by either of the IRES RNAs however, C11 treatment markedly reduced hnRNP A1 binding. The effects of C11 on the translational state of the cyclin D1 and c-MYC mRNAs were additionally examined. Polysome analysis was performed and as shown in FIG. 1E, C11 treatment induced a significant shift in both cyclin D1 and c-MYC mRNA to monosomal/nonribosomal fractions while actin mRNA distribution was unaffected. This is consistent with previous observations that actin mRNA is translated via cap-dependent initiation (26). C11 did not appear to alter cyclin D1 or c-MYC steady-state mRNA levels as total monosomal/nonribosomal plus polysomal mRNA content was unchanged as compared controls suggesting that the inhibitor does not affect transcription or mRNA stability. As hnRNP A1 is also a splicing factor, it was determined whether C11 affected Delta Max splicing in GBM. EGFRvIII signaling promotes Delta Max splicing via hnRNP A1 in GBM (22) and as shown in FIG. 1F (top panel), C11 treatment did not alter Delta Max splicing in U87 cells stably expressing EGFRvIII. To further confirm that C11 does not affect eIF-4Emediated initiation eIF-4E was immunoprecipitated from cells treated with C11 and assessed the relative amounts of both cyclin D1 and c-MYC mRNAs within these complexes by qRT-PCR. As shown in FIG. 1F (middle panel), C11 exposure did not affect either cyclin D1 or c-MYC association with eIF-4E. Finally, cyclin D1 and c-MYC protein levels following C11 exposure in LN229 and SF763 cells were markedly reduced (FIG. 1F, bottom panel). These data demonstrate that C11 inhibits both cyclin D1 and c-MYC IRES-mediated mRNA translation leading to reductions in protein levels.

### Example 4: C11 Inhibits mTOR Inhibitor-Induced IRES Activity and Potentiates PP242 Anti-GBM Responses

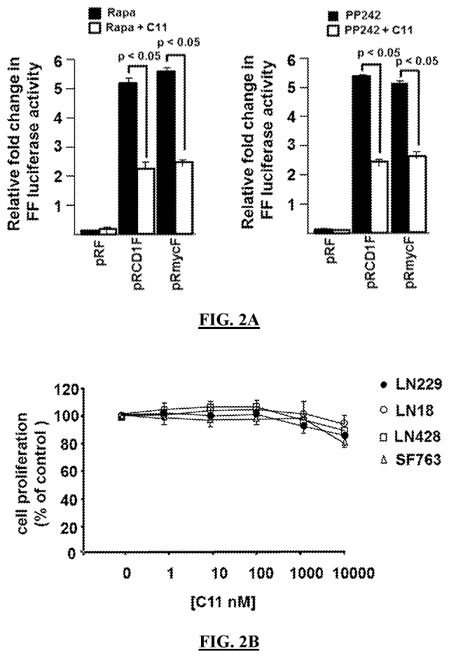

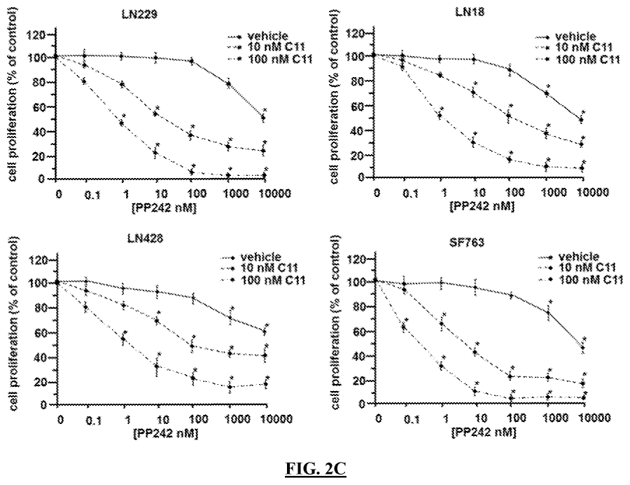

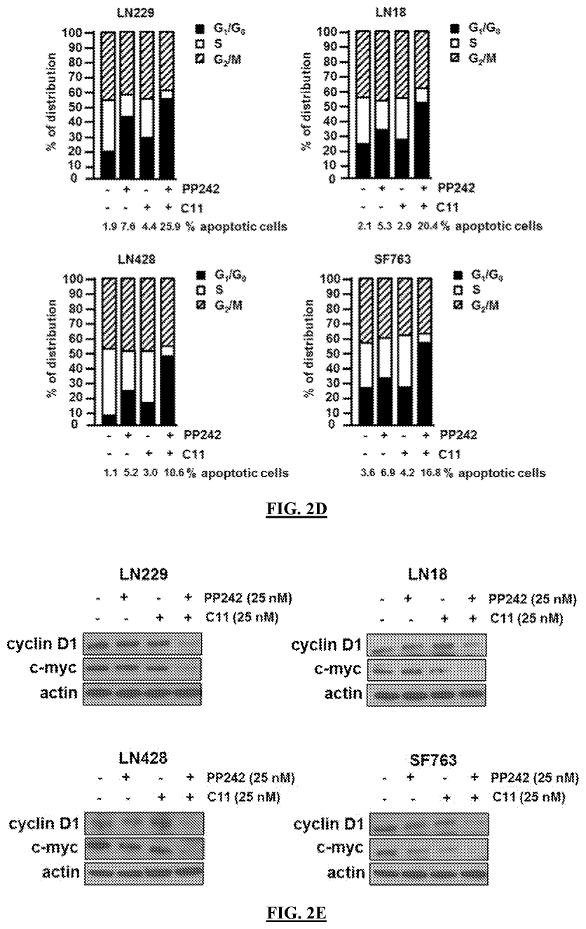

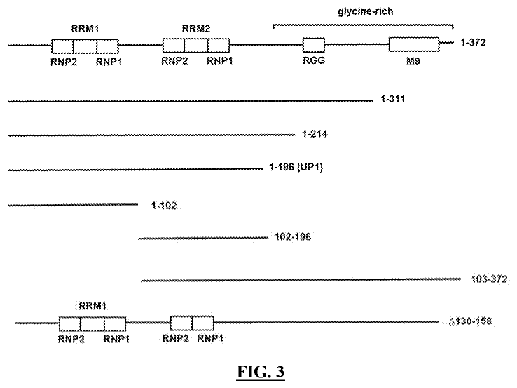

FIG. 2 shows (A) Inhibition of mTOR inhibitor-induced IRES activity in LN229 cells. Cells transiently transfected with the indicated IRES mRNA reporter constructs were treated with rapamycin or rapamycin+C11 (left panel), PP242 or PP242+C11 (right panel) and luciferase activities determined. Results are expressed as relative fold change in firefly (FF) luciferase activity and the mean and +S.D. are shown for three independent experiments. (B) Growth inhibition of GBM cell lines following 48 h culture in C11. Data represent mean±S.D. of three independent experiments. (C) Combination analysis of PP242 and C11 inhibitors in GBM cell lines treated with the indicated doses of PP242 alone or in combination for 48 h, and percent growth relative to control cultures was assessed via XTT assays. Control cell were treated with DMSO vehicle. Data are means±S.D., n=3. (D) Cell-cycle phase distributions were determined on the indicated GBM cell lines in the absence or presence of PP242 or C11 as shown. Percent apoptotic cells as determined via Annexin V staining are also shown below each graph. One of three experiments with similar results is shown.

mTOR inhibitors induce the upregulation of IRES activity as an intrinsic mechanism of resistance to this class of inhibitors, it was investigated whether C11 would enhance PP242 cytotoxicity. As shown in FIG. 2A, treatment with rapamycin (left panel) or PP242 (right panel) led to dramatic induction of cyclin D1 and c-MYC IRES activity which was significantly inhibited upon cotreatment with C11. No significant inhibition of cell growth was observed from C11 treatment at any of the concentrations tested up to 10 μM in several GBM lines (FIG. 2B). This was similar to previous findings with multiple myeloma cell lines (20). However, as shown in FIG. 2C, in LN229, LN18, LN428 and SF763 GBM lines, treatment with C11 at 10 and 100 nM concentrations resulted in synergistic inhibition of cell growth over a wide range of PP242 concentrations tested (CI=0.5 at ED50 ratio of 1:100; (28,29)). It was also determined whether the combination of C11 with PP242 induced G1 arrest and apoptosis in the four GBM cells lines. As shown in FIG. 2D, PP242 treatment increased G1 arrest and cotreatment with C11 markedly stimulated G1 arrest. Similarly, PP242 alone induced the percentage of apoptotic cells and when combined with C11 further potentiated the percentage of cells undergoing apoptosis. These results demonstrate that C11 significantly enhances PP242 induced G1 arrest and apoptosis in GBM lines.

### Example 5: Structure-Activity Relationship Studies Derive Active Analogs of C11

Table 3 shows compounds of the invention and a summary of PP242 synergistic anti-GBM activities for the synthesized analogs in each series. Fold decrease in either PP242-induced cyclin D1 or c-MYC IRES activity in LN229 relative to values obtained with the parent compound C11 are shown for each analog. For each analog the combination index (CI) was calculated from combination analyses performed with PP242 and analog, as in FIG. 2C and as described in (28). CI=1.0 (dose additive), CI<0.5 (synergy), CI<0.3 (strong synergy). Percent apoptosis was determined for LN229 cells cotreated with PP242 (50 nM) and analog (100 nM) at 24 h via Annexin V staining.

The structure-activity relationships (SAR) for C11 and related compounds were investigated by synthesizing and testing the analogs listed in Table 1. Four series of analogs were synthesized focused on modifying specific regions of C11 as shown in Table 1. Each analog was tested for its ability to decrease PP242-induced cyclin D1 and c-MYC IRES activity. Additionally, it was determined whether the analogs demonstrated synergistic cytotoxic responses when combined with PP242 in LN229 cells. The degree of apoptotic cell death following co-treatment with analog and PP242 was also monitored. These results are summarized in Table 3.

TABLE 3
SAR Relationships for C11

| | | Cyclin D1 | c-myc | CI ED50, | % Apoptosis |
|---|---|---|---|---|---|
| Compound | Structure | (decrease) | (decrease) | 1:100 | (100 nM) |

Series A

| | | | | | |
|---|---|---|---|---|---|
| C11 | 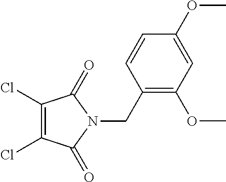 [SMILES: COc1ccc(CN2C(=O)C(Cl)=C(Cl)C2=O)c(OC)c1] | 1.0 | 1.0 | 0.50 | 25.9 |
| IRES-J000 | 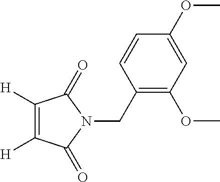 [SMILES: COc1ccc(CN2C(=O)C=CC2=O)c(OC)c1] | 1.05 ± 0.21 | 1.01 ± 0.03 | 0.51 | 25.6 |
| IRES-J004 | 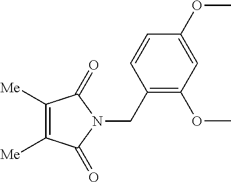 [SMILES: COc1ccc(CN2C(=O)C(C)=C(C)C2=O)c(OC)c1] | 1.42 ± 0.07 | 1.39 ± 0.11 | 0.82 | 24.8 |
| IRES-J005 | 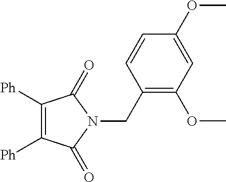 [SMILES: COc1ccc(CN2C(=O)C(c3ccccc3)=C(c3ccccc3)C2=O)c(OC)c1] | 1.32 ± 0.14 | 1.28 ± 0.16 | 0.91 | 23.5 |
| IRES-J006 | 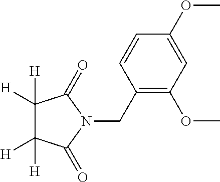 [SMILES: COc1ccc(CN2C(=O)CCC2=O)c(OC)c1] | 1.26 ± 0.10 | 1.25 ± 0.19 | 0.94 | 20.1 |
| IRES-J007 | 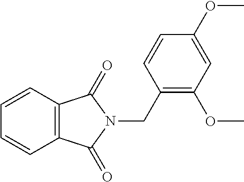 [SMILES: COc1ccc(CN2C(=O)c3ccccc3C2=O)c(OC)c1] | 4.97 ± 0.13 | 6.51 ± 0.07 | 0.42 | 62.4 |

Series B

| | | | | | |
|---|---|---|---|---|---|
| IRES-J000 | 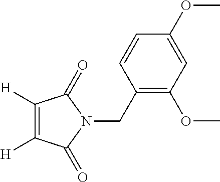 [SMILES: COc1ccc(CN2C(=O)C=CC2=O)c(OC)c1] | 1.05 ± 0.21 | 1.01 ± 0.03 | 0.51 | 25.6 |
| IRES-J001 | 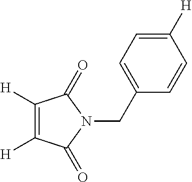 [SMILES: O=C1C=CC(=O)N1Cc1ccccc1] | 1.49 ± 0.05 | 1.12 ± 0.13 | 0.89 | 30.2 |
| IRES-J002 | 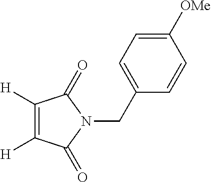 [SMILES: COc1ccc(CN2C(=O)C=CC2=O)cc1] | 1.60 ± 0.18 | 1.37 ± 0.21 | 0.86 | 28.6 |
| IRES-J003 | 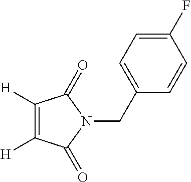 [SMILES: O=C1C=CC(=O)N1Cc1ccc(F)cc1] | 0.97 ± 0.06 | 1.19 ± 0.04 | 0.88 | 27.1 |
| IRES-J008 | 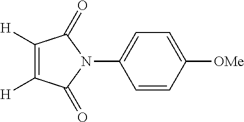 [SMILES: COc1ccc(N2C(=O)C=CC2=O)cc1] | 5.58 ± 0.16 | 5.92 ± 0.10 | 0.39 | 75.7 |

Series C

| | | | | | |
|---|---|---|---|---|---|
| IRES-J009 | 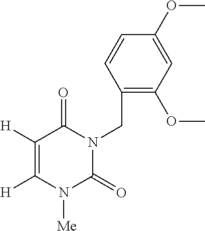 [SMILES: COc1ccc(Cn2c(=O)ccn(C)c2=O)c(OC)c1] | 2.57 ± 0.17 | 3.19 ± 0.09 | 0.33 | 67.9 |
| IRES-J016 | 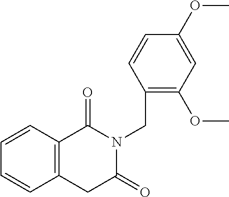 [SMILES: COc1ccc(CN2C(=O)Cc3ccccc3C2=O)c(OC)c1] | 2.63 ± 0.11 | 1.90 ± 0.16 | 0.62 | 21.4 |
| IRES-J017 | 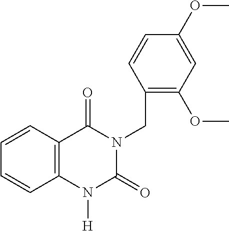 [SMILES: COc1ccc(Cn2c(=O)[nH]c3ccccc3c2=O)c(OC)c1] | 2.75 ± 0.13 | 2.21 ± 0.24 | 0.69 | 20.4 |
| IRES-J018 | 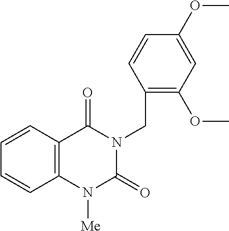 [SMILES: COc1ccc(Cn2c(=O)c3ccccc3n(C)c2=O)c(OC)c1] | 1.52 ± 0.29 | 1.73 ± 0.20 | 0.89 | 19.7 |
| IRES-J019 | 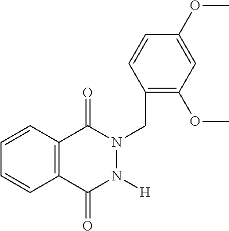 [SMILES: COc1ccc(Cn2[nH]c(=O)c3ccccc3c2=O)c(OC)c1] | 1.42 ± 0.09 | 1.01 ± 0.26 | 0.81 | 21.5 |
| IRES-J020 | 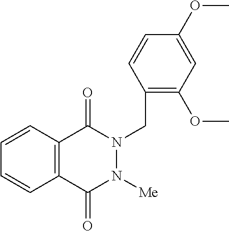 [SMILES: COc1ccc(Cn2c(=O)c3ccccc3c(=O)n2C)c(OC)c1] | 1.08 ± 0.12 | 1.06 ± 0.18 | 0.93 | 19.3 |

Series D

| | | | | | |
|---|---|---|---|---|---|
| IRES-J010 | 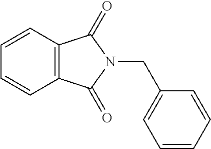 [SMILES: O=C1c2ccccc2C(=O)N1Cc1ccccc1] | 1.77 ± 0.16 | 1.94 ± 0.25 | 0.83 | 19.7 |
| IRES-J011 | 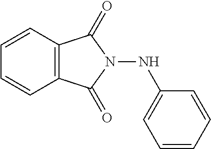 [SMILES: O=C1c2ccccc2C(=O)N1Nc1ccccc1] | 1.91 ± 0.1 | 1.64 ± 0.18 | 0.86 | 22.8 |
| IRES-J012 | 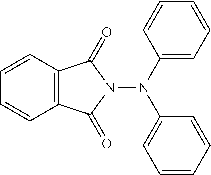 [SMILES: O=C1c2ccccc2C(=O)N1N(c1ccccc1)c1ccccc1] | 1.02 ± 0.31 | 1.53 ± 0.17 | 0.89 | 20.1 |
| IRES-J013 | 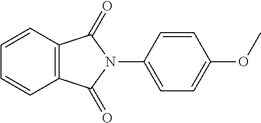 [SMILES: COc1ccc(N2C(=O)c3ccccc3C2=O)cc1] | 1.17 ± 0.21 | 1.72 ± 0.06 | 0.85 | 19.2 |
| IRES-J014 | 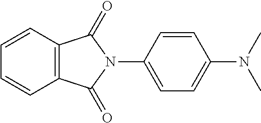 [SMILES: CN(C)c1ccc(N2C(=O)c3ccccc3C2=O)cc1] | 1.86 ± 0.27 | 1.33 ± 0.11 | 0.84 | 20.6 |
| IRES-J015 | 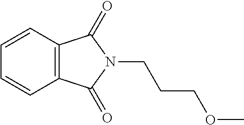 [SMILES: COCCCN1C(=O)c2ccccc2C1=O] | 1.61 ± 0.15 | 1.25 ± 0.24 | 0.88 | 18.3 |

In the series A analogs, IRES-J007, with a phthalimido group in place of the dichloromaleimide unit, demonstrated the greatest degree of IRES inhibition relative to C11. The inhibition of IRES activity correlated with an increase in synergistic antitumor response in combination with PP242 (reduction in combination index; CI value) and a marked induction of apoptosis. Additional modifications of the IRES-J007 analog were synthesized; however none of these compounds exhibited significantly improved properties compared to C11. Within the series B and C analogs, IRES-J008, with a 4-methoxyphenyl substituent in place of the 2,4-dimethoxybenzyl unit, and to a lesser degree, IRES-J009, with an N1-methyluracil unit in place of the dichloromaleimide unit, inhibited cyclin D1 and c-MYC PP242-induced IRES activity. Both of these analogs also demonstrated significant synergistic cytotoxic effects in combination with PP242 with coordinate induction of apoptosis. The in vitro cytotoxicities of these three analogs relative to C11 in human neurons were also determined. IRES-J007 displayed the least toxicity to normal neurons with no significant cytotoxic effects for concentrations up to 10 mM and was therefore chosen for further study. The reduced toxicity of the IRES-J007 versus C11 might possible be due to the lack of the quite reactive dichloromaleimide unit present in C11 which is absent in IRES-J007, or may be due to other factors.

### Example 6: C11 or IRES-J007 Blocks Association of UP1 to Cyclin D1 or c-MYC IRESs

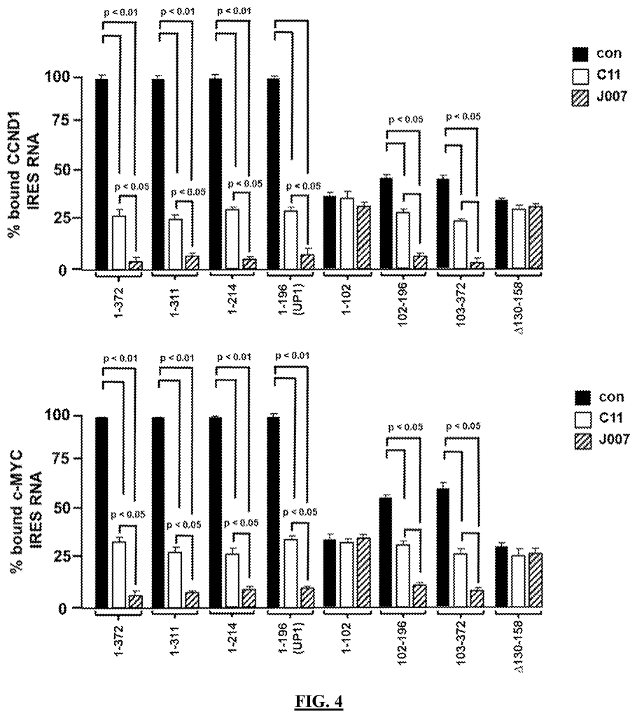

To begin to investigate the mechanism of action of C11 and IRES-J007, it was initially determined whether the UP1 fragment of hnRNP A1 was sufficient to recapitulate C11 or IRESJ007-mediated inhibition of IRES binding to this ITAF. Several GST-tagged deletion mutants of hnRNP A1 were generated and purified as shown in FIG. 4A. The relative association between the mutant proteins and either the cyclin D1 or c-MYC IRESs was determined by filter binding assays in the absence or presence of the inhibitors (FIG. 4B). C-terminal deletion of the glycine-rich region, encompassing the RGG box and M9 domain did not affect either cyclin D1 or c-MYC IRES binding and binding was inhibitable by C11. The UP1 fragment (a.a. 1-196) containing RRM1 and RRM2 and immediately adjacent sequences efficiently bound the IRESs and either C11 or IRES-J007 blocked association. The analog IRES-J007 demonstrated a modest, yet significant increase in its ability to block UP1/IRES RNA interactions compared to C11. These results were consistent with the relative improvement of the analog to inhibit PP242-induced IRES activity (Table 3). A mutant encompassing the first 102 amino acids and containing only RRM1 of hnRNP A1 did not demonstrate IRES binding however, mutant 102-196, containing RRM2 did bind both the cyclin D and c-MYC IRESs and both interactions were sensitive to C11 or IRES-J007. Additionally, a mutant encompassing residues 103-372, containing RRM2 bound both IRES sequences and binding was reduced in the presence of either of the inhibitors. Next, only RRM2 (a.a. 130-158) was removed from hnRNP A1 and this mutant did not bind either of the IRES RNAs. These data suggest that much of the C-terminal half of hnRNP A1 is dispensable and that RRM1 alone is insufficient to mediate efficient IRES binding and inhibition by C11 or IRES-J007. The presence of RRM2 is necessary and appears to cooperate with RRM1 to mediate IRES binding that is sensitive to C₁₁ or IRES-J007. Finally, UP1 is capable of IRES binding that is blocked by either C11 or the analog IRES-J007.

### Example 7: C11 or the Analog IRES-J007 Bind to a Small Pocket Structure within UP1

FIG. 4 shows (A) Schematic representation of the various hnRNP A1 deletion mutations. Mutant 1-196 constitutes the Up1 fragment of full-length human hnRNP A1. In the Δ130-158 mutant, the sequences encompassing RRM2 have been removed. (B) Binding of either cyclin D1 (top panel) or c-MYC (bottom panel) IRES RNAs to GST-tagged hnRNP A1 mutants in the absence or presence of C11 or IRES-J007 as assayed by filter binding. The mean and +SD are shown for three independent experiments.

FIG. 5 shows (A) In silico docking analysis was utilized to predict potential binding sites for C11 and IRES-J007 on UP1. The configurations with the most favorable binding energies were visualized using PyMOL v1.5.6. The electrostatic surface representation of the crystal structure of UP1 is shown with RNP residues of RRM1 and RRM2 labeled in blue. In the 90°-rotated model, the inhibitor interaction pocket is shown in yellow. The inset is a close-up of C11 and IRES-J007 binding to the potential binding site on UP1. Residues predicted to interact with the inhibitors are labeled. (B) Purified GST-tagged wild-type hnRNP A1 (A1) and mutant A1 (4ΔA1) proteins harboring alanine substitutions at all four potential binding sites (120, 123,124 and 171) were added to uncross-linked, C11 and J007-cross-linked beads. Isolated wild-type (A1) and mutant (4ΔA1) proteins were resolved by SDS-PAGE and silver-stained to monitor purity (top panels). The binding of A1 to control, C11 and J007 beads was detected by immunoblotting with GST antibodies (bottom panel). (C) Inhibition of basal IRES activity in 293T cells upon treatment with C11 or IRES-J007. IRES reporter activity was assessed following 24 h treatment with 50 nM concentrations of each inhibitor as indicated. The mean and +SD are shown for three independent experiments. (D) RNA-pull down assays utilizing biotinylated cyclin D1 or c-MYC IRES RNAs of 293T cell extracts treated with the inhibitors as in FIG. 2C.

To further examine how C11 and IRES-J007 inhibit hnRNP A1 function, used in silico docking analysis was used to create a potential model for the binding of these inhibitors to UP1. To generate unbiased predictive virtual docking models, the crystal structure of monomeric UP1 was obtained from the Protein Data Bank (PDB) and docking studies were performed using AutoDock Vina molecular modeling simulation software (24). Two potential binding sites were identified, however the binding models which predicted the highest binding free energy (ΔG) occupied the site shown in FIG. 5A. This binding site was in close proximity to RRM2 and predicted binding to both C11 and IRES-J007 with similar binding free energies. The interaction maps for this binding site revealed that four residues (H120, D123, Y124 and N171) were predominantly involved in the interaction with C11 or IRESJ007. C11 and IRES-J007 occupied the same pocket with the configurations shown in FIG. 5A (inset) displaying the best binding scores, ΔG of −8.09 and −9.26 kcal/mol, respectively. To confirm the accuracy of this binding model, C11 and IRES-J007 crosslinked affinity beads were generated using a photo-cross-linking procedure (25). Subsequently the inhibitor-coupled beads (C11 beads or J007 beads; FIG. 5B) were tested for whether they bound to native or mutant hnRNP A1 proteins harboring alanine substitutions at all four potential residues predicted to participate in the interactions. Recombinant GST-tagged native hnRNP A1 (A1) and mutant A1 proteins (4ΔA1) were purified by glutathione affinity methods. The purity was confirmed by SDS-PAGE followed by silver staining (FIG. 5B, upper panels). The purified proteins were then incubated with control, C11 or J007 beads and binding analyzed by immunoblotting using anti-GST antibodies. Native GST-tagged hnRNP A1 bound to either C11 or J007 beads, but not to control beads; however, the amount of mutant hnRNP A1 (4ΔA1) which bound either C11 or J007 beads was markedly reduced relative to native hnRNP A1 (FIG. 5B, lower panel; see also FIG. 5C). These results suggest that C11 and IRES-J007 bind hnRNP A1 through the residues predicted by the interaction model. It was also determined whether C11 or the analog IRES-J007 would inhibit basal cyclin D1 or c-MYC IRES activity in 293T cells which express high endogenous levels of hnRNP A1 and show elevated IRES activity (30). As shown in FIG. 5D, 293T cells transiently transfected with the cyclin D1 and c-MYC IRES mRNA reporters and subsequently treated with either the C11 or IRES-J007 analog demonstrated reduced IRES activity. IRES-J007 inhibited cyclin D1 and c-MYC IRES activity to a greater extent as compared to the parent compound C11. RNA-pull down assays in 293T cells also demonstrated an improved ability of the analog IRES-J007 to block cyclin D1 or c-MYC IRES-hnRNP A1 interactions relative to C11 (FIG. 5E).

### Example 8: In Vivo Effects of IRES-J007 and PP242 Combination Therapy in Xenografts

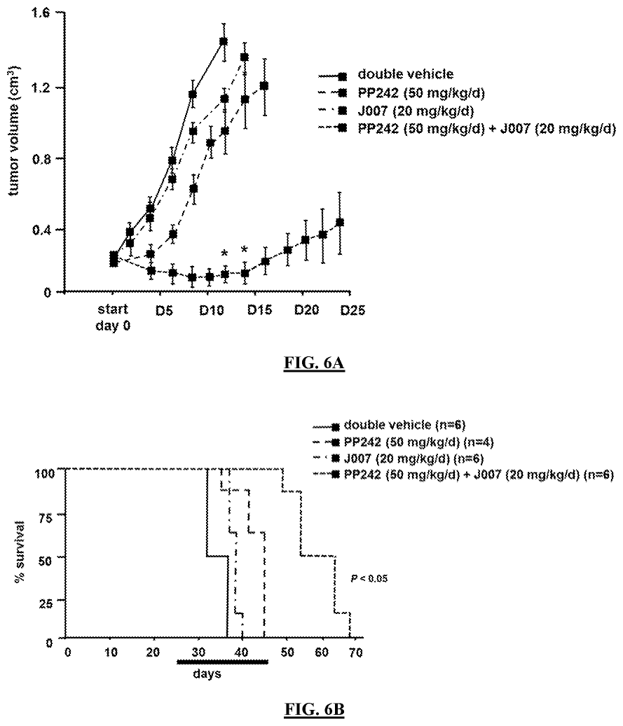

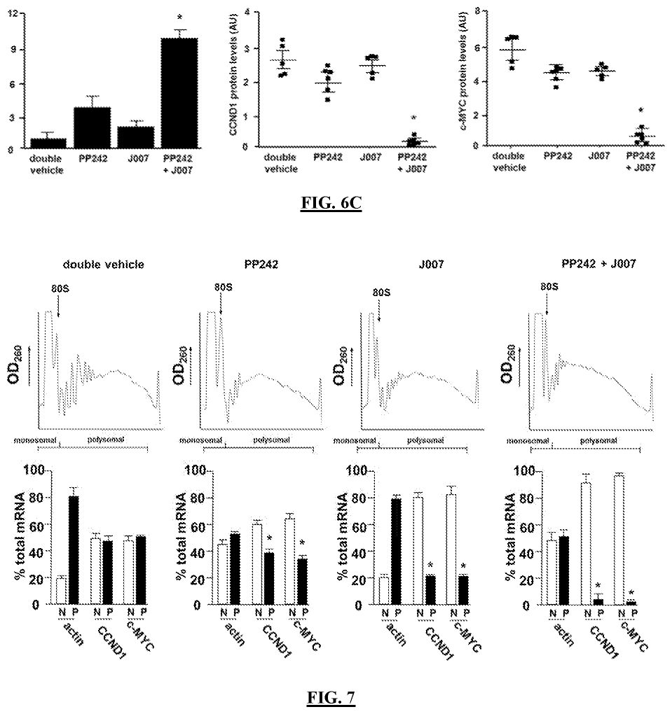

FIG. 6 shows (A). Tumor burden of SCID mice implanted with LN229 cells and treated double vehicle, PP242, J007, or combination for ten consecutive days and tumor growth assessed every two days following initiation of treatment (start, day 0). *, P<0.05, significantly different from double vehicle, PP242 (50 mg/kg/d) and J007 (20 mg/kg/d). (B). Overall survival of subcutaneous LN229 tumors receiving the indicated treatment schedules. (C). Left panel, apoptotic cells were identified by TUNEL assays of sections prepared from harvested tumors at day 12 following initiation of treatment regimens. Data are expressed as the number of positive apoptotic bodies divided by high power field (hpf; 10-12 hpf/tumor). Values are means+S.D., *, P<0.05. Middle panel, Cyclin D1 protein levels in tumors. Values are means±S.D., *, P<0.05, significantly different from vehicle, PP242 and J007. Right panel, c-MYC protein levels in tumors. Values are means±S.D., *, P<0.05, significantly different from vehicle, PP242 and J007. To determine whether the combination of IRES and mTOR inhibitor cotherapy would be efficacious in vivo, xenograft studies were conducted utilizing LN229 cells in mice. Mice were subcutaneously implanted with tumor cells and once tumors were palpable and reached ˜200 mm3 in size, mice were randomized into treatment groups receiving double vehicle, PP242 (50 mg/kg/d), IRES-J007 (20 mg/kg/d) and PP242 (50 mg/kg/d)+IRES-J007 (20 mg/kg/d). As shown in FIG. 6A, xenografts receiving monotherapy with PP242 resulted in significant inhibition of tumor growth rate (36% inhibition at end of dosing period; tumor growth delay, 6.0 days). Tumor growth following monotherapy with IRES-J007 did not differ significantly and exhibited similar growth rates to double vehicle controls consistent with the lack of effects of this inhibitor alone in vitro. However, the combination of PP242 and IRESJ007 was significantly more efficacious then either monotherapies alone (93% inhibition at end of dosing period; tumor growth delay, 20.5 days). Consistent with the effects on xenograft growth, overall survival of mice receiving combination IRES and mTOR therapy was significantly extended as compared to either of the monotherapies (FIG. 6B). Notably, mice tolerated this dosing regimen without obvious short or long-term toxicity or weight loss. The induction of apoptosis was also monitored via TUNEL staining of tumor section from harvested tumors upon autopsy. As can be seen in FIG. 6C (left panel), significant staining was observed in tumors which received combination therapy, corroborating the increases in apoptotic cell death observed in vitro (see Table 3). Marked reductions in cyclin D1 and c-MYC protein levels were also displayed in tumors receiving combination therapy (FIG. 6C, center and right panels).

### Example 9: Cyclin D1 and c-MYC mRNA Translational State in Response to IRES and mTOR Inhibitors

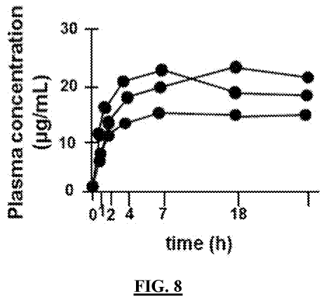

FIG. 7 shows Polysome distributions of cyclin D1, c-MYC and actin mRNAs from xenografted tumors harvested from mice receiving the indicated treatment schedules. Tumor extracts were subjected to sucrose density gradient centrifugation, fractionated and pooled into nonribosomal, monosomal fraction (N, white bars) and a polysomal fraction (P, black bars). Purified RNAs were used in real-time qRT-PCR analysis to determine the distributions of cyclin D1, c-MYC and actin mRNAs across the gradients. Polysome gradient tracings are shown above each graph. Means and +S.D. values are shown for quadruplicate RT-PCR measurements. *, P<0.05.

Cyclin D1 and c-MYC IRES activity nearly exclusively directs mRNA translation of these determinants following mTOR inhibitor exposure (18). To discern whether alterations in cyclin D1 and c-MYC expression mediated by the inhibitor therapies in xenografted tumors were the result of actual changes in mRNA translational efficiency of these transcripts, polysome analysis of freshly harvested LN229 tumors was conducted following the last day of inhibitor dosing. Polysomes were separated via sucrose density gradient sedimentation and fractionated into heavy polysomal and nonribosomal/monosomal fractions. Spectrophoretic monitoring of fractions at 260 nm was used to identify polysomal and nonribosomal containing fractions and monitor polysome integrity as before (FIG. 1E). As shown in FIG. 7, tumors from mice which received double vehicle treatments cyclin D1 and c-MYC were present in polysomal fractions at approximately 45% and 50% of total cyclin D1 and c-MYC mRNA, respectively. Mice which received PP242 monotherapy exhibited significantly different cyclin D1 and c-MYC mRNA translational states, reduced to 38% and 35%, respectively. Actin mRNA polysomal distribution was also monitored and as shown this mRNA, whose synthesis is mediated via eIF-4E dependent initiation, was markedly redistributed to nonribosomal/polysomal fractions demonstrating effective inhibition of cap-dependent initiation. Mice that received IRES-J007 monotherapy displayed a significant reduction in cyclin D1 and c-MYC mRNA translational efficiency consistent with the results of inhibiting IRES-mediated translation via C11 treatment in vitro (see FIG. 1E). Actin mRNA translational efficiency was unaffected in tumors treated with IRES-J007 monotherapy. However, tumors which received PP242 and IRES-J007 cotherapy displayed a larger reduction in both cyclin D1 and c-MYC translational efficiency compared to IRES-J007 or PP242 monotherapy, with most of these transcripts being redistributed to nonribosomal/monosomal fractions (CCND1, 5% polysomal; c-MYC 3% polysomal, *=P<0.05). These data taken together, suggest that the IRES-J007 inhibitor effectively inhibits cyclin D1 and c-MYC IRES-mediated protein synthesis in these tumors.

### Example 10: Pharmacokinetic Properties of IRES-J007

The pharmacokinetic properties of IRES-J007 in mice were determined. FIG. 8 shows the plasma concentration of IRES-J007 following oral gavage administration in three different mice at 20 mg/kg. IRES-J007 was ˜50% bioavailable following oral delivery with a serum half-life of approximately 30 hours. Trough levels observed following a single 20 mg/kg dose exceeded concentrations expected to block hnRNP A1 IRES activity based on data obtained from glioblastoma cell line studies. The pharmacokinetic parameters are provided in Table 4:

TABLE 4
Pharmacokinetic Parameters for IRES-J007

| Route | IV | PO |
|---|---|---|
| AUC (h · μg/mL) | 1294.1 | 759.2 |
| Half-life (h) | 13.6¹ | 27.4² |
| Clearance (L/h · kg) | 9.1 | 30.2² |
| Cₘₐₓ (μg/mL) | 284.5 | 17.2 |
| Tmax (h) | 0.05 | 7.2 |
| Relative Bioavailability | 100% | 40.1%³ |

¹T_{1/2}^{b},
²T_{1/2}^{K10},
³Bioavailablity calculated based on AUC

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

### REFERENCES

1. Dunn, G. P., Rinne, M. L., Wykosky, J., Genovese, G., Quayle, S. N., Dunn, I. F., Agarwalla, P. K., Chheda, M. G., Campos, B., Wang, A., Brennan, C., Ligon, K. L., Furnari, F., Cavenee, W. K., Depinho, R. A., Chin, L., and Hahn, W. C. (2012) Emerging insights into the molecular and cellular basis of glioblastoma. Genes Dev 26, 756-784
2. Stupp, R., Mason, W. P., van den Bent, M. J., Weller, M., Fisher, B., Taphoorn, M. J., Belanger, K., Brandes, A. A., Marosi, C., Bogdahn, U., Curschmann, J., Janzer, R. C., Ludwin, S. K., Gorlia, T., Allgeier, A., Lacombe, D., Cairncross, J. G., Eisenhauer, E., Mirimanoff, R. O., European Organisation for, R., Treatment of Cancer Brain, T., Radiotherapy, G., and National Cancer Institute of Canada Clinical Trials, G. (2005) Radiotherapy plus concomitant and adjuvant temozolomide for glioblastoma. N Engl J Med 352, 987-996
3. Cancer Genome Atlas Research, N. (2008) Comprehensive genomic characterization defines human glioblastoma genes and core pathways. Nature 455, 1061-1068
4. Parsons, D. W., Jones, S., Zhang, X., Lin, J. C., Leary, R. J., Angenendt, P., Mankoo, P., Carter, H., Siu, I. M., Gallia, G. L., Olivi, A., McLendon, R., Rasheed, B. A., Keir, S., Nikolskaya, T., Nikolsky, Y., Busam, D. A., Tekleab, H., Diaz, L. A., Jr., Hartigan, J., Smith, D. R., Strausberg, R. L., Marie, S. K., Shinjo, S. M., Yan, H., Riggins, G. J., Bigner, D. D., Karchin, R., Papadopoulos, N., Parmigiani, G., Vogelstein, B., Velculescu, V. E., and Kinzler, K. W. (2008) An integrated genomic analysis of human glioblastoma multiforme. Science 321, 1807-1812
5. Fan, Q. W., and Weiss, W. A. (2010) Targeting the RTK-PI3K-mTOR axis in malignant glioma: overcoming resistance. Curr Top Microbiol Immunol 347, 279-296
6. Cloughesy, T. F., Cavenee, W. K., and Mischel, P. S. (2014) Glioblastoma: from molecular pathology to targeted treatment. Annu Rev Pathol 9, 1-25
7. Gini, B., Zanca, C., Guo, D., Matsutani, T., Masui, K., Ikegami, S., Yang, H., Nathanson, D., Villa, G. R., Shackelford, D., Zhu, S., Tanaka, K., Babic, I., Akhavan, D., Lin, K., Assuncao, A., Gu, Y., Bonetti, B., Mortensen, D. S., Xu, S., Raymon, H. K., Cavenee, W. K., Furnari, F. B., James, C. D., Kroemer, G., Heath, J. R., Hege, K., Chopra, R., Cloughesy, T. F., and Mischel, P. S. (2013) The mTOR kinase inhibitors, CC214-1 and CC214-2, preferentially block the growth of EGFRvIII-activated glioblastomas. Clin Cancer Res 19, 5722-5732
8. Laplante, M., and Sabatini, D. M. (2012) mTOR signaling in growth control and disease. Cell 149, 274-293
9. Betz, C., and Hall, M. N. (2013) Where is mTOR and what is it doing there? J Cell Biol 203, 563-574
10. Cloughesy, T. F., Yoshimoto, K., Nghiemphu, P., Brown, K., Dang, J., Zhu, S., Hsueh, T., Chen, Y., Wang, W., Youngkin, D., Liau, L., Martin, N., Becker, D., Bergsneider, M., Lai, A., Green, R., Oglesby, T., Koleto, M., Trent, J., Horvath, S., Mischel, P. S., Mellinghoff, I. K., and Sawyers, C. L. (2008) Antitumor activity of rapamycin in a Phase I trial for patients with recurrent PTEN-deficient glioblastoma. PLoS Med 5, e8
11. Chang, S. M., Wen, P., Cloughesy, T., Greenberg, H., Schiff, D., Conrad, C., Fink, K., Robins, H. I., De Angelis, L., Raizer, J., Hess, K., Aldape, K., Lamborn, K. R., Kuhn, J., Dancey, J., Prados, M. D., North American Brain Tumor, C., and the National Cancer, I. (2005) Phase II study of CCI-779 in patients with recurrent glioblastoma multiforme. Invest New Drugs 23, 357-361
12. Wu, S. H., Bi, J. F., Cloughesy, T., Cavenee, W. K., and Mischel, P. S. (2014) Emerging function of mTORC2 as a core regulator in glioblastoma: metabolic reprogramming and drug resistance. Cancer Biol Med 11, 255-263
13. Masri, J., Bernath, A., Martin, J., Jo, O. D., Vartanian, R., Funk, A., and Gera, J. (2007) mTORC2 activity is elevated in gliomas and promotes growth and cell motility via overexpression of rictor. Cancer Res 67, 11712-11720
14. Iwanami, A., Gini, B., Zanca, C., Matsutani, T., Assuncao, A., Nael, A., Dang, J., Yang, H., Zhu, S., Kohyama, J., Kitabayashi, I., Cavenee, W. K., Cloughesy, T. F., Furnari, F. B., Nakamura, M., Toyama, Y., Okano, H., and Mischel, P. S. (2013) PML mediates glioblastoma resistance to mammalian target of rapamycin (mTOR)-targeted therapies. Proc Natl Acad Sci USA 110, 4339-4344
15. Benavides-Serrato, A., Anderson, L., Holmes, B., Cloninger, C., Artinian, N., Bashir, T., and Gera, J. (2014) mTORC2 modulates feedback regulation of p38 MAPK activity via DUSP10/MKP5 to confer differential responses to PP242 in glioblastoma. Genes Cancer 5, 393-406
16. Tanaka, K., Babic, I., Nathanson, D., Akhavan, D., Guo, D., Gini, B., Dang, J., Zhu, S., Yang, H., De Jesus, J., Amzajerdi, A. N., Zhang, Y., Dibble, C. C., Dan, H., Rinkenbaugh, A., Yong, W. H., Vinters, H. V., Gera, J. F., Cavenee, W. K., Cloughesy, T. F., Manning, B. D., Baldwin, A. S., and Mischel, P. S. (2011) Oncogenic EGFR signaling activates an mTORC2-NF-kappaB pathway that promotes chemotherapy resistance. Cancer Discov 1, 524-538
17. Shi, Y., Sharma, A., Wu, H., Lichtenstein, A., and Gera, J. (2005) Cyclin D1 and c-myc internal ribosome entry site (IRES)-dependent translation is regulated by AKT activity and enhanced by rapamycin through a p38 MAPK- and ERK-dependent pathway. J Biol Chem 280, 10964-10973
18. Martin, J., Masri, J., Cloninger, C., Holmes, B., Artinian, N., Funk, A., Ruegg, T., Anderson, L., Bashir, T., Bernath, A., Lichtenstein, A., and Gera, J. (2011) Phosphomimetic substitution of heterogeneous nuclear ribonucleoprotein A1 at serine 199 abolishes AKT-dependent internal ribosome entry site-transacting factor (ITAF) function via effects on strand annealing and results in mammalian target of rapamycin complex 1 (mTORC1) inhibitor sensitivity. J Biol Chem 286, 16402-16413
19. Jo, O. D., Martin, J., Bernath, A., Masri, J., Lichtenstein, A., and Gera, J. (2008) Heterogeneous nuclear ribonucleoprotein A1 regulates cyclin D1 and c-myc internal ribosome entry site function through Akt signaling. J Biol Chem 283, 23274-23287
20. Cloninger, C., Bernath, A., Bashir, T., Holmes, B., Artinian, N., Ruegg, T., Anderson, L., Masri, J., Lichtenstein, A., and Gera, J. (2011) Inhibition of SAPK2/p38 enhances sensitivity to mTORC1 inhibition by blocking IRES-mediated translation initiation in glioblastoma. Mol Cancer Ther 10, 2244-2256
21. Shi, Y., Yang, Y., Hoang, B., Bardeleben, C., Holmes, B., Gera, J., and Lichtenstein, A. (2015) Therapeutic potential of targeting IRES-dependent c-myc translation in multiple myeloma cells during ER stress. Oncogene
22. Culjkovic, B., Topisirovic, I., Skrabanek, L., Ruiz-Gutierrez, M., and Borden, K. L. (2005) eIF4E promotes nuclear export of cyclin D1 mRNAs via an element in the 3′UTR. J Cell Biol 169, 245-256
23. Babic, I., Anderson, E. S., Tanaka, K., Guo, D., Masui, K., Li, B., Zhu, S., Gu, Y., Villa, G. R., Akhavan, D., Nathanson, D., Gini, B., Mareninov, S., Li, R., Camacho, C. E., Kurdistani, S. K., Eskin, A., Nelson, S. F., Yong, W. H., Cavenee, W. K., Cloughesy, T. F., Christofk, H. R., Black, D. L., and Mischel, P. S. (2013) EGFR mutation-induced alternative splicing of Max contributes to growth of glycolytic tumors in brain cancer. Cell Metab 17, 1000-1008
24. Bonnal, S., Pileur, F., Orsini, C., Parker, F., Pujol, F., Prats, A. C., and Vagner, S. (2005) Heterogeneous nuclear ribonucleoprotein A1 is a novel internal ribosome entry site trans-acting factor that modulates alternative initiation of translation of the fibroblast growth factor 2 mRNA. J Biol Chem 280, 4144-4153
25. Trott, O., and Olson, A. J. (2010) AutoDock Vina: improving the speed and accuracy of docking with a new scoring function, efficient optimization, and multithreading. J Comput Chem 31, 455-461
26. Kanoh, N., Honda, K., Simizu, S., Muroi, M., and Osada, H. (2005) Photo-cross-linked small-molecule affinity matrix for facilitating forward and reverse chemical genetics. Angew Chem Int Ed Engl 44, 3559-3562
27. Thompson, S. R. (2012) So you want to know if your message has an IRES? Wiley Interdiscip Rev RNA 3, 697-705
28. Thoreen, C. C., Chantranupong, L., Keys, H. R., Wang, T., Gray, N. S., and

Sabatini, D. M. (2012) A unifying model for mTORC1-mediated regulation of mRNA translation. Nature 485, 109-113
29. Chou, T. C. (2010) Drug combination studies and their synergy quantification using the Chou-Talalay method. Cancer Res 70, 440-446
30. Chou, T. C., and Talalay, P. (1984) Quantitative analysis of dose-effect relationships: the combined effects of multiple drugs or enzyme inhibitors. Adv Enzyme Regul 22, 27-55
31. Michlewski, G., and Caceres, J. F. (2010) Antagonistic role of hnRNP A1 and KSRP in the regulation of let-7a biogenesis. Nat Struct Mol Biol 17, 1011-1018
32. Dreyfuss, G., Kim, V. N., and Kataoka, N. (2002) Messenger-RNA-binding proteins and the messages they carry. Nat Rev Mol Cell Biol 3, 195-205
33. Lewis, S. M., and Holcik, M. (2008) For IRES trans-acting factors, it is all about location. Oncogene 27, 1033-1035
34. Lewis, S. M., Veyrier, A., Hosszu Ungureanu, N., Bonnal, S., Vagner, S., and Holcik, M. (2007) Subcellular relocalization of a trans-acting factor regulates XIAP IRES-dependent translation. Mol Biol Cell 18, 1302-1311
35. Jean-Philippe, J., Paz, S., and Caputi, M. (2013) hnRNP A1: the Swiss army knife of gene expression. Int J Mol Sci 14, 18999-19024
36. Morgan, C. E., Meagher, J. L., Levengood, J. D., Delproposto, J., Rollins, C., Stuckey, J. A., and Tolbert, B. S. (2015) The First Crystal Structure of the UP1 Domain of hnRNP A1 Bound to RNA Reveals a New Look for an Old RNA Binding Protein. J Mol Biol 427, 3241-3257
37. Ko, C. C., Chen, Y. J., Chen, C. T., Liu, Y. C., Cheng, F. C., Hsu, K. C., and Chow, L. P. (2014) Chemical proteomics identifies heterogeneous nuclear ribonucleoprotein (hnRNP) A1 as the molecular target of quercetin in its anti-cancer effects in PC-3 cells. J Biol Chem 289, 22078-22089
38. Fridell, R. A., Truant, R., Thorne, L., Benson, R. E., and Cullen, B. R. (1997) Nuclear import of hnRNP A1 is mediated by a novel cellular cofactor related to karyopherin-beta. J Cell Sci 110 (Pt 11), 1325-1331
39. Rebane, A., Aab, A., and Steitz, J. A. (2004) Transportins 1 and 2 are redundant nuclear import factors for hnRNP A1 and HuR. RNA 10, 590-599

1. A compound having the structure of formula I or a pharmaceutically acceptable salt or prodrug thereof: 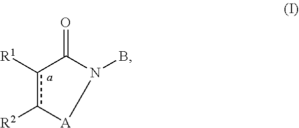
wherein:
A is selected from —C(O)—, —C(O)C(R³)₂—, —NR⁴C(O)—, or —C(O)NR⁴—, wherein the right-hand valence is attached to the nitrogen atom of Formula I;
B is selected from alkyl, aryl, heteroaryl, aralkyl, heteroaralkyl, or arylamino, or heteroarylamino;
R¹ and R² are independently selected from H, alkyl, cycloalkyl, heterocyclyl, aryl, heteroaryl, cyano, halo, hydroxyl, carbonyl, thiocarbonyl, alkoxyl, amino, amido, amidine, imine, sulfhydryl, alkylthio, sulfate, sulfonate, sulfamoyl, sulfonamido, or sulfonyl; or R′ and R², taken together with the carbon atoms that separate them, complete a cycle or heterocycle having from 4 to 8 atoms in the ring structure;
the bond marked with an ‘a’ is selected from a single or double bond;
R³ is selected from H, alkyl, cycloalkyl, heterocyclyl, aryl, heteroaryl, cyano, halo, hydroxyl, carbonyl, thiocarbonyl, alkoxyl, amino, amido, amidine, imine, sulfhydryl, alkylthio, sulfate, sulfonate, sulfamoyl, sulfonamido, or sulfonyl; and
R⁴ is selected from H, alkyl, cycloalkyl, heterocyclyl, aryl, heteroaryl, carbonyl, thiocarbonyl, or alkoxyl.

2. The compound of claim 1, wherein R¹ and R² are independently selected from H, alkyl, phenyl, or fluoro.

3. The compound of claim 1, wherein R′ and R², taken together with the carbon atoms that separate them, complete a cyclic or heterocyclic moiety having from 4 to 8 atoms in the ring structure; and further wherein the cyclic or heterocyclic moiety is optionally substituted by at least one alkyl, cycloalkyl, heterocyclyl, aryl, heteroaryl, cyano, halo, hydroxyl, carbonyl, thiocarbonyl, alkoxyl, amino, amido, amidine, imine, sulfhydryl, alkylthio, sulfate, sulfonate, sulfamoyl, sulfonamido, or sulfonyl.

4. The compound of claim 1, wherein:
R³ is alkyl; and
R⁴ is alkyl.

5. The compound of claim 1, wherein the compound is represented by one of the following structures:

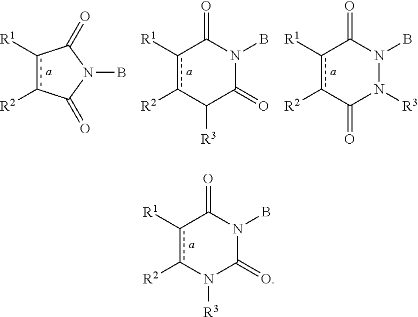

6. The compound of claim 1, wherein the compound is represented by one of the following structures:

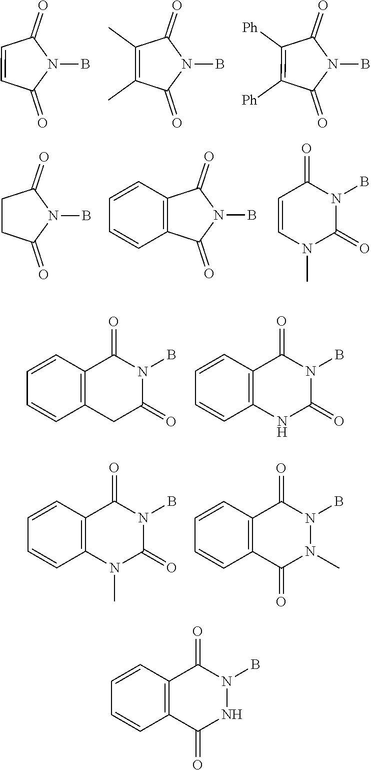

7. The compound of claim 1, wherein B is selected from aryl, heteroaryl, aralkyl, heteroaralkyl, arylamino, or heteroarylamino.

8. The compound of claim 1, wherein B is selected from phenyl, benzyl, phenylamino, and diphenylamino.

9. The compound of claim 1, wherein B is unsubstituted or is substituted with alkyl, alkoxy, halo, or amino.

10. The compound of claim 9, wherein B is selected from unsubstituted benzyl, 2,4-dimethoxybenzyl, 4-methoxybenzyl, 4-fluorobenzyl, 4-methoxyphenyl, unsubstituted phenylamino, unsubstituted diphenylamino, or 4-dimethylaminophenyl.

11. The compound of claim 1, wherein the compound of Formula I is selected from IRES-J007, IRES-J008, or IRES-J009.

12. A pharmaceutical composition comprising the compound of claim 1 and a pharmaceutically acceptable excipient.

13. The pharmaceutical composition of claim 12, further comprising an mTOR inhibitor.

14-17. (canceled)

18. A method of treating a mammal suffering from cancer, comprising administering a compound or composition of claim 1.

19. The method of claim 18, wherein the method further comprises conjointly administering an mTOR inhibitor.

20. The method of claim 19, wherein the mTOR inhibitor is selected from rapamycin or PP242.

21. The method of claim 20, wherein the cancer is ovarian cancer, endometrial cancer, breast cancer, colon cancer, brain cancer, neuroblastoma, lung cancer, skin cancer, renal cancer, liver cancer, prostate cancer, head or neck carcinoma, pancreatic cancer, thyroid cancer, leukemia; lymphoma, multiple myeloma, rhabdomyosarcoma, osteosarcoma, or Ewing sarcoma.

22. The method of claim 21, wherein the cancer is glioblastoma.

23. A method of inhibiting IRES-mediated protein synthesis within a cell, comprising contacting the cell with a compound of claim 1.

24. (canceled)

25. The method of claim 23, further comprising contacting the cell with an mTOR inhibitor.
